SEQUENCE LISTING (65 sequences)

SEQ ID NO: 1 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 3 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 4 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 5 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 6 - protein, 347 aa, Homo sapiens
MALLFSLILAICTRPGFLASPSGVRLVGGLHRCEGRVEVEQKGQWGTVCDDGWDIKDVAVLCRELGCGAASGTPSGILYEPPAEKEQKVLIQSVSCTGTEDTLAQCEQEEVYDCSHDEDAGASCENPESSFSPVPEGVRLADGPGHCKGRVEVKHQNQWYTVCQTGWSLRAAKVVCRQLGCGRAVLTQKRCNKHAYGRKPIWLSQMSCSGREATLQDCPSGPWGKNTCNHDEDTWVECEDPFDLRLVGGDNLCSGRLEVLHKGVWGSVCDDNWGEKEDQVVCKQLGCGKSLSPSFRDRKCYGPGVGRIWLDNVRCSGEEQSLEQCQHRFWGFHDCTHQEDVAVICSG

SEQ ID NO: 7 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 8 - protein, 222 aa, Homo sapiens
MKLPLLLALLFGAVSALHLRSETSTFETPLGAKTLPEDEETPEQEMEETPCRELEEEEEWGSGSEDASKKDGAVESISVPDMVDKNLTCPEEEDTVKVVGIPGCQTCRYLLVRSLQTFSQAWFTCRRCYRGNLVSIHNFNINYRIQCSVSALNQGQVWIGGRITGSGRCRRFQWVDGSRWNFAYWAAHQPWSRGGHCVALCTRGGYWRRAHCLRRLPFICSY

SEQ ID NO: 9 - protein, 472 aa, Homo sapiens
MATKCGNCGPGYSTPLEAMKGPREEIVYLPCIYRNTGTEAPDYLATVDVDPKSPQYCQVIHRLPMPNLKDELHHSGWNTCSSCFGDSTKSRTKLVLPSLISSRIYVVDVGSEPRAPKLHKVIEPKDIHAKCELAFLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPDAAQGFVGCALSSTIQRFYKNEGGTWSVEKVIQVPPKKVKGWLLPEMPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWDKQFYPDLIREGSVMLQVDVDTVKGGLKLNPNFLVDFGKEPLGPALAHELRYPGGDCSSDIWI

SEQ ID NO: 10 - protein, 481 aa, Homo sapiens
MGALARALPSILLALLLTSTPEALGANPGLVARITDKGLQYAAQEGLLALQSELLRITLPDFTGDLRIPHVGRGRYEFHSLNIHSCELLHSALRPVPGQGLSLSISDSSIRVQGRWKVRKSFFKLQGSFDVSVKGISISVNLLLGSESSGRPTVTASSCSSDIADVEVDMSGDLGWLLNLFHNQIESKFQKVLESRICEMIQKSVSSDLQPYLQTLPVTTEIDSFADIDYSLVEAPRATAQMLEVMFKGEIFHRNHRSPVTLLAAVMSLPEEHNKMVYFAISDYVFNTASLVYHEEGYLNFSITDDMIPPDSNIRLTTKSFRPFVPRLARLYPNMNLELQGSVPSAPLLNFSPGNLSVDPYMEIDAFVLLPSSSKEPVFRLSVATNVSATLTFNTSKITGFLKPGKVKVELKESKVGLFNAELLEALLNYYILNTFYPKFNDKLAEGFPLPLLKRVQLYDLGLQIHKDFLFLGANVQYMRV

SEQ ID NO: 11 - protein, 2322 aa, Homo sapiens [sequence omitted]

SEQ ID NO: 12 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 13 - protein, 201 aa, Homo sapiens
MTEKAPEPHVEEDDDDELDSKLNYKPPPQKSLKELQEMDKDDESLIKYKKTLLGDGPVVTDPKAPNVVVTRLTLVCESAPGPITMDLTGDLEALKKETIVLKEGSEYRVKIHFKVNRDIVSGLKYVQHTYRTGVKVDKATFMVGSYGPRPEEYEFLTPVEEAPKGMLARGTYHNKSFFTDDDKQDHLSWEWNLSIKKEWTE

SEQ ID NO: 14 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 15 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 16 - protein, 1685 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 17 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 18 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 19 - protein, 816 aa, Homo sapiens
MESRVLLRTFCLIFGLGAVWGLGVDPSLQIDVLTELELGESTTGVRQVPGLHNGTKAFLFQDTPRSIKASTATAEQFFQKLRNKHEFTILVTLKQTHLNSGVILSIHHLDHRYLELESSGHRNEVRLHYRSGSHRPHTEVFPYILADDKWHKLSLAISASHLILHIDCNKIYERVVEKPSTDLPLGTTFWLGQRNNAHGYFKGIMQDVQLLVMPQGFIAQCPDLNRTCPTCNDFHGLVQKIMELQDILAKTSAKLSRAEQRMNRLDQCYCERTCTMKGTTYREFESWIDGCKNCTCLNGTIQCETLICPNPDCPLKSALAYVDGKCCKECKSICQFQGRTYFEGERNTVYSSSGVCVLYECKDQTMKLVESSGCPALDCPESHQITLSHSCCKVCKGYDFCSERHNCMENSICRNLNDRAVCSCRDGFRALREDNAYCEDIDECAEGRHYCRENTMCVNTPGSFMCICKTGYIRIDDYSCTEHDECITNQHNCDENALCFNTVGGHNCVCKPGYTGNGTTCKAFCKDGCRNGGACIAANVCACPQGFTGPSCETDIDECSDGFVQCDSRANCINLPGWYHCECRDGYHDNGMFSPSGESCEDIDECGTGRHSCANDTICFNLDGGYDCRCPHGKNCTGDCIHDGKVKHNGQIWVLENDRCSVCSCQNGFVMCRRMVCDCENPTVDLFCCPECDPRLSSQCLHQNGETLYNSGDTWVQNCQQCRCLQGEVDCWPLPCPDVECEFSILPENECCPRCVTDPCQADTIRNDITKTCLDEMNVVRFTGSSWIKHGTECTLCQCKNGHICCSVDPQCLQEL

SEQ ID NO: 20 - protein, 172 aa, Homo sapiens
MVGPAPRRRLRPLAALALVLALAPGLPTARAGQTPRPAERGPPVRLFTEEELARYGGEEEDQPIYLAVKGVVFDVTSGKEFYGRGAPYNALTGKDSTRGVAKMSLDPADLTHDTTGLTAKELEALDEVFTKVYKAKYPIVGYTARRILNEDGSPNLDFKPEDQPHFDIKDEF

SEQ ID NO: 21 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 22 - protein, 156 aa, Homo sapiens
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 23 - protein, 383 aa, Homo sapiens
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDGGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 24 - protein, 457 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 25 - protein, 421 aa, Homo sapiens
MGFLSPIYVIFFFFGVKVHCQYETYQWDEDYDQEPDDDYQTGFPFRQNVDYGVPFHQYTLGCVSECFCPTNFPSSMYCDNRKLKTIPNIPMHIQQLYLQFNEIEAVTANSFINATHLKEINLSHNKIKSQKIDYGVFAKLPNLLQLHLEHNNLEEFPFPLPKSLERLLLGYNEISKLQTNAMDGLVNLTMLDLCYNYLHDSLLKDKIFAKMEKLMQLNLCSNRLESMPPGLPSSLMYLSLENNSISSIPEKYFDKLPKLHTLRMSHNKLQDIPYNIFNLPNIVELSVGHNKLKQAFYIPRNLEHLYLQNNEIEKMNLTVMCPSIDPLHYHHLTYIRVDQNKLKEPISSYIFFCFPHIHTIYYGEQRSTNGQTIQLKTQVFRRFPDDDDESEDHDDPDNAHESPEQEGAEGHFDLHYYENQE

SEQ ID NO: 26 - protein, 1366 aa, Homo sapiens [sequence omitted]

SEQ ID NO: 27 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 28 - protein, 1609 aa, Homo sapiens [sequence omitted]

SEQ ID NO: 29 - protein, 1786 aa, Homo sapiens
MGLLQLLAFSFLALCRARVRAQEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGNCFCYGHASECAPVDGFNEEVEGMVHGHCMCRHNTKGLNCELCMDFYHDLPWRPAEGRNSNACKKCNCNEHSISCHFDMAVYLATGNVSGGVCDDCQHNTMGRNCEQCKPFYYQHPERDIRDPNFCERCTCDPAGSQNEGICDSYTDFSTGLIAGQCRCKLNVEGEHCDVCKEGFYDLSSEDPFGCKSCACNPLGTIPGGNPCDSETGHCYCKRLVTGQHCDQCLPEHWGLSNDLDGCRPCDCDLGGALNNSCFAESGQCSCRPHMIGRQCNEVEPGYYFATLDHYLYEAEEANLGPGVSIVERQYIQDRIPSWTGAGFVRVPEGAYLEFFIDNIPYSMEYDILIRYEPQLPDHWEKAVITVQRPGRIPTSSRCGNTIPDDDNQVVSLSPGSRYVVLPRPVCFEKGTNYTVRLELPQYTSSDSDVESPYTLIDSLVLMPYCKSLDIFTVGGSGDGVVTNSAWETFQRYRCLENSRSVVKTPMTDVCRNIIFSISALLHQTGLACECDPQGSLSSVCDPNGGQCQCRPNVVGRTCNRCAPGTFGFGPSGCKPCECHLQGSVNAFCNPVTGQCHCFQGVYARQCDRCLPGHWGFPSCQPCQCNGHADDCDPVTGECLNCQDYTMGHNCERCLAGYYGDPIIGSGDHCRPCPCPDGPDSGRQFARSCYQDPVTLQLACVCDPGYIGSRCDDCASGYFGNPSEVGGSCQPCQCHNNIDTTDPEACDKETGRCLKCLYHTEGEHCQFCRFGYYGDALRQDCRKCVCNYLGTVQEHCNGSDCQCDKATGQCLCLPNVIGQNCDRCAPNTWQLASGTGCDPCNCNAAHSFGPSCNEFTGQCQCMPGFGGRTCSECQELFWGDPDVECRACDCDPRGIETPQCDQSTGQCVCVEGVEGPRCDKCTRGYSGVFPDCTPCHQCFALWDVIIAELTNRTHRFLEKAKALKISGVIGPYRETVDSVERKVSEIKDILAQSPAAEPLKNIGNLFEEAEKLIKDVTEMMAQVEVKLSDTTSQSNSTAKELDSLQTEAESLDNTVKELAEQLEFIKNSDIRGALDSITKYFQMSLEAEERVNASTTEPNSTVEQSALMRDRVEDVMMERESQFKEKQEEQARLLDELAGKLQSLDLSAAAEMTCGTPPGASCSETECGGPNCRTDEGERKCGGPGCGGLVTVAHNAWQKAMDLDQDVLSALAEVEQLSKMVSEAKLRADEAKQSAEDILLKTNATKEKMDKSNEELRNLIKQIRNFLTQDSADLDSIEAVANEVLKMEMPSTPQQLQNLTEDIRERVESLSQVEVILQHSAADIARAEMLLEEAKRASKSATDVKVTADMVKEALEEAEKAQVAAEKAIKQADEDIQGTQNLLTSIESETAASEETLFNASQRISELERNVEELKRKAAQNSGEAEYIEKVVYTVKQSAEDVKKTLDGELDEKYKKVENLIAKKTEESADARRKAEMLQNEAKTLLAQANSKLQLLKDLERKYEDNQRYLEDKAQELARLEGEVRSLLKDISQKVAVYSTCL

SEQ ID NO: 30 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 31 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 32 - protein, 227 aa, Homo sapiens
MRVAGAAKLVVAVAVFLLTFYVISQVFEIKMDASLGNLFARSALDTAARSTKPPRYKCGISKACPEKHFAFKMASGAANVVGPKICLEDNVLMSGVKNNVGRGINVALANGKTGEVLDTKYFDMWGGDVAPFIEFLKAIQDGTIVLMGTYDDGATKLNDEARRLIADLGSTSITNLGFRDNWVFCGGKGIKTKSPFEQHIKNNKDTNKYEGWPEVVEMEGCIPQKQD

SEQ ID NO: 33 - protein, 892 aa, Homo sapiens
MDHYDSQQTNDYMQPEEDWDRDLLLDPAWEKQQRKTFTAWCNSHLRKAGTQIENIEEDFRDGLKLMLLLEVISGERLAKPERGKMRVHKISNVNKALDFIASKGVKLVSIGAEEIVDGNVKMTLGMIWTIILRFAIQDISVEETSAKEGLLLWCQRKTAPYKNVNIQNFHISWKDGLGFCALIHRHRPELIDYGKLRKDDPLTNLNTAFDVAEKYLDIPKMLDAEDIVGTARPDEKAIMTYVSSFYHAFSGAQKAETAANRICKVLAVNQENEQLMEDYEKLASDLLEWIRRTIPWLENRVPENTMHAMQQKLEDFRDYRRLHKPPKVQEKCQLEINFNTLQTKLRLSNRPAFMPSEGRMVSDINNAWGCLEQVEKGYEEWLLNEIRRLERLDHLAEKFRQKASIHEAWTDGKEAMLRQKDYETATLSEIKALLKKHEAFESDLAAHQDRVEQIAAIAQELNELDYYDSPSVNARCQKICDQWDNLGALTQKRREALERTEKLLETIDQLYLEYAKRAAPFNNWMEGAMEDLQDTFIVHTIEEIQGLTTAHEQFKATLPDADKERLAILGIHNEVSKIVQTYHVNMAGTNPYTTITPQEINGKWDHVRQLVPRRDQALTEEHARQQHNERLRKQFGAQANVIGPWIQTKMEEIGRISIEMHGTLEDQLSHLRQYEKSIVNYKPKIDQLEGDHQLIQEALIFDNKHTNYTMEHIRVGWEQLLTTIARTINEVENQILTRDAKGISQEQMNEFRASFNHFDRDHSGTLGPEEFKACLISLGYDIGNDPQGEAEFARIMSIVDPNRLGVVTFQAFIDFMSRETADTDTADQVMASFKILAGDKNYITMDELRRELPPDQAEYCIARMAPYTGPDSVPGALDYMSFSTALYGESDL

SEQ ID NO: 34 - protein, 286 aa, Homo sapiens
MADFDDRVSDEEKVRIAAKFITHAPPGEFNEVFNDVRLLLNNDNLLREGAAHAFAQYNMDQFTPVKIEGYEDQVLITEHGDLGNSRFLDPRNKISFKFDHLRKEASDPQPEEADGGLKSWRESCDSALRAYVKDHYSNGFCTVYAKTIDGQQTIIACIESHQFQPKNFWNGRWRSEWKFTITPPTAQVVGVLKIQVHYYEDGNVQLVSHKDVQDSLTVSNEAQTAKEFIKIIENAENEYQTAISENYQTMSDTTFKALRRQLPVTRTKIDWNKILSYKIGKEMQNA

SEQ ID NO: 35 - protein, 967 aa, Homo sapiens
MAKGFYISKSLGILGILLGVAAVCTIIALSVVYSQEKNKNANSSPVASTTPSASATTNPASATTLDQSKAWNRYRLPNTLKPDSYRVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 36 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 37 - protein, 442 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 38 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 39 - protein, 718 aa, Homo sapiens
MCASVKYNIRGPALIPRMKTKHRIYYITLFSIVLLGLIATGMFQFWPHSIESSNDWNVEKRSIRDVPVVRLPADSPIPERGDLSCRMHTCFDVYRCGFNPKNKIKVYIYALKKYVDDFGVSVSNTISREYNELLMAISDSDYYTDDINRACLFVPSIDVLNQNTLRIKETAQAMAQLSRWDRGTNHLLFNMLPGGPPDYNTALDVPRDRALLAGGGFSTWTYRQGYDVSIPVYSPLSAEVDLPEKGPGPRQYFLLSSQVGLHPEYREDLEALQVKHGESVLVLDKCTNLSEGVLSVRKRCHKHQVFDYPQVLQEATFCVVLRGARLGQAVLSDVLQAGCVPVVIADSYILPFSEVLDWKRASVVVPEEKMSDVYSILQSIPQRQIEEMQRQARWFWEAYFQSIKAIALATLQIINDRIYPYAAISYEEWNDPPAVKWGSVSNPLFLPLIPPQSQGFTAIVLTYDRVESLFRVITEVSKVPSLSKLLVVWNNQNKNPPEDSLWPKIRVPLKVVRTAENKLSNRFFPYDEIETEAVLAIDDDIIMLTSDELQFGYEVWREFPDRLVGYPGRLHLWDHEMNKWKYESEWTNEVSMVLTGAAFYHKYFNYLYTYKMPGDIKNWVDAHMNCEDIAMNFLVANVTGKAVIKVTPRKKFKCPECTAIDGLSLDQTHMVERSECINKFASVFGTMPLKVVEHRADPVLYKDDFPEKLKSFPNIGSL

SEQ ID NO: 40 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 41 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 42 - protein, 149 aa, Homo sapiens
MARSNLPLALGLALVAFCLLALPRDARARPQERMVGELRDLSPDDPQVQKAAQAAVASYNMGSNSIYYFRDTHIIKAQSQLVAGIKYFLTMEMGSTDCRKTRVTGDHVDLTTCPLAAGAQQEKLRCDFEVLVVPWQNSSQLLKHNCVQM

SEQ ID NO: 43 - protein, 485 aa, Homo sapiens
MSVPLLKIGVVLSTMAMITNWMSQTLPSLVGLNTTKLSAAGGGTLDRSTGVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKAIKAKMDELRPLIPVLEEYKADAKLVLQFKEEVQNLTSVLNELQEEIGAYDYDELQSRVSNLEERLRACMQKLACGKLTGISDPVTVKTSGSRFGSWMTDPLAPEGDNRVWYMDGYHNNRFVREYKSMVDFMNTDNFTSHRLPHPWSGTGQVVYNGSIYFNKFQSHIIIRFDLKTETILKTRSLDYAGYNNMYHYAWGGHSDIDLMVDESGLWAVYATNQNAGNIVVSRLDPVSLQTLQTWNTSYPKRSAGEAFIICGTLYVTNGYSGGTKVHYAYQTNASTYEYIDIPFQNKYSHISMLDYNPKDRALYAWNNGHQILYNVTLFHVIRSDEL

SEQ ID NO: 44 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 45 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 46 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 47 - protein, 605 aa, Homo sapiens
MALRKGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNVAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC

SEQ ID NO: 48 - protein, 586 aa, Homo sapiens
MPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLERQQLETEKKRRETVEREKEQMMREKEELMLRLQDYEEKTKKAERELSEQIQRALQLEEERKRAQEEAERLEADRMAALRAKEELERQAVDQIKSQEQLAAELAEYTAKIALLEEARRRKEDEVEEWQHRAKEAQDDLVKTKEELHLVMTAPPPPPPPVYEPVSYHVQESLQDEGAEPTGYSAELSSEGIRDDRNEEKRITEAEKNERVQRQLLTLSSELSQARDENKRTHNDIIHNENMRQGRDKYKTLRQIRQGNTKQRIDEFEAL

SEQ ID NO: 49 - protein, 127 aa, Homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

SEQ ID NO: 50 - protein, 1346 aa, Homo sapiens
MKSPRRTTLCLMFIVIYSSKAALNWNYESTIHPLSLHEHEPAGEEALRQKRAVATKSPTAEEYTVNIEISFENASFLDPIKAYLNSLSFPIHGNNTDQITDILSINVTTVCRPAGNEIWCSCETGYGWPRERCLHNLICQERDVFLPGHHCSCLKELPPNGPFCLLQEDVTLNMRVRLNVGFQEDLMNTSSALYRSYKTDLETAFRKGYGILPGFKGVTVTGFKSGSVVVTYEVKTTPPSLELIHKANEQVVQSLNQTYKMDYNSFQAVTINESNFFVTPEIIFEGDTVSLVCEKEVLSSNVSWRYEEQQLEIQNSSRFSIYTALFNNMTSVSKLTIHNITPGDAGEYVCKLILDIFEYECKKKIDVMPIQILANEEMKVMCDNNPVSLNCCSQGNVNWSKVEWKQEGKINIPGTPETDIDSSCSRYTLKADGTQCPSGSSGTTVIYTCEFISAYGARGSANIKVTFISVANLTITPDPISVSEGQNFSIKCISDVSNYDEVYWNTSAGIKIYQRFYTTRRYLDGAESVLTVKTSTREWNGTYHCIFRYKNSYSIATKDVIVHPLPLKLNIMVDPLEATVSCSGSHHIKCCIEEDGDYKVTFHMGSSSLPAAKEVNKKQVCYKHNFNASSVSWCSKTVDVCCHFTNAANNSVWSPSMKLNLVPGENITCQDPVIGVGEPGKVIQKLCRFSNVPSSPESPIGGTITYKCVGSQWEEKRNDCISAPINSLLQMAKALIKSPSQDEMLPTYLKDLSISIDKAEHEISSSPGSLGAIINILDLLSTVPTQVNSEMMTHVLSTVNVILGKPVLNTWKVLQQQWTNQSSQLLHSVERFSQALQSGDSPPLSFSQTNVQMSSTVIKSSHPETYQQRFVFPYFDLWGNVVIDKSYLENLQSDSSIVTMAFPTLQAILAQDIQENNFAESLVMTTTVSHNTTMPFRISMTFKNNSPSGGETKCVFWNFRLANNTGGWDSSGCYVEEGDGDNVTCICDHLTSFSILMSPDSPDPSSLLGILLDIISYVGVGFSILSLAACLVVEAVVWKSVTKNRTSYMRHTCIVNIAASLLVANTWFIVVAAIQDNRYILCKTACVAATFFIHFFYLSVFFWMLTLGLMLFYRLVFILHETSRSTQKAIAFCLGYGCPLAISVITLGATQPREVYTRKNVCWLNWEDTKALLAFAIPALIIVVVNITITIVVITKILRPSIGDKPCKQEKSSLFQISKSIGVLTPLLGLTWGFGLTTVFPGTNLVFHIIFAILNVFQGLFILLFGCLWDLKVQEALLNKFSLSRWSSQHSKSTSLGSSTPVFSMSSPISRRFNNLFGKTGTYNVSTPEATSSSLENSSSASSLLN

SEQ ID NO: 51 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLPTKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 52 - protein, 545 aa, Homo sapiens
MPPFLLLTCLFITGTSVSPVALDPCSAYISLNEPWRNTDHQLDESQGPPLCDNHVNGEWYHFTGMAGDAMPTFCIPENHCGTHAPVWLNGSHPLEGDGIVQRQACASFNGNCCLWNTTVEVKACPGGYYVYRLTKPSVCFHVYCGHFYDICDEDCHGSCSDTSECTCAPGTVLGPDRQTCFDENECEQNNGGCSEICVNLKNSYRCECGVGRVLRSDGKTCEDVEGCHNNNGGCSHSCLGSEKGYQCECPRGLVLSEDNHTCQVPVLCKSNAIEVNIPRELVGGLELFLTNTSCRGVSNGTHVNILFSLKTCGTVVDVVNDKIVASNLVTGLPKQTPGSSGDFIIRTSKLLIPVTCEFPRLYTISEGYVPNLRNSPLEIMSRNHGIFPFTLEIFKDNEFEEPYREALPTLKLRDSLYFGIEPVVHVSGLESLVESCFATPTSKIDEVLKYYLIRDGCVSDDSVKQYTSRDHLAKHFQVPVFKFVGKDHKEVFLHCRVLVCGVLDERSRCAQGCHRRMRRGAGGEDSAGLQGQTLTGGPIRIDWED

SEQ ID NO: 53 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 54 - protein, 480 aa, Homo sapiens
MRFVVALVLLNVAAAGAVPLLATESVKQEEAGVRPSAGNVSTHPSLSQRPGGSTKSHPEPQTPKDSPSKSSAEAQTPEDTPNKSGAEAKTQKDSSNKSGAEAKTQKGSTSKSGSEAQTTKDSTSKSHPELQTPKDSTGKSGAEAQTPEDSPNRSGAEAKTQKDSPSKSGSEAQTTKDVPNKSGADGQTPKDGSSKSGAEDQTPKDVPNKSGAEKQTPKDGSNKSGAEEQGPIDGPSKSGAEEQTSKDSPNKVVPEQPSRKDHSKPISNPSDNKELPKADTNQLADKGKLSPHAFKTESGEETDLISPPQEEVKSSEPTEDVEPKEAEDDDTGPEEGSPPKEEKEKMSGSASSENREGTLSDSTGSEKDDLYPNGSGNGSAESSHFFAYLVTAAILVAVLYIAHHNKRKIIAFVLEGKRSKVTRRPKASDYQRLDQKYVLILNVFPAPPKRSFLPQVLTEWYIPLEKDERHQWIVLLSFQL

SEQ ID NO: 55 - protein, 623 aa, Homo sapiens
MESYHKPDQQKLQALKDTANRLRISSIQATTAAGSGHPTSCCSAAEIMAVLFFHTMRYKSQDPRNPHNDRFVLSKGHAAPILYAVWAEAGFLAEAELLNLRKISSDLDGHPVPKQAFTDVATGSLGQGLGAACGMAYTGKYFDKASYRVYCLLGDGELSEGSVWEAMAFASIYKLDNLVAILDINRLGQSDPAPLQHQMDIYQKRCEAFGWHAIIVDGHSVEELCKAFGQAKHQPTAIIAKTFKGRGITGVEDKESWHGKPLPKNMAEQIIQEIYSQIQSKKKILATPPQEDAPSVDIANIRMPSLPSYKVGDKIATRKAYGQALAKLGHASDRIIALDGDTKNSTFSEIFKKEHPDRFIECYIAEQNMVSIAVGCATRNRTVPFCSTFAAFFTRAFDQIRMAAISESNINLCGSHCGVSIGEDGPSQMALEDLAMFRSVPTSTVFYPSDGVATEKAVELAANTKGICFIRTSRPENAIIYNNNEDFQVGQAKVVLKSKDDQVTVIGAGVTLHEALAAAELLKKEKINIRVLDPFTIKPLDRKLILDSARATKGRILTVEDHYYEGGIGEAVSSAVVGEPGITVTHLAVNRVPRSGKPAELLKMFGIDRDAIAQAVRGLITKA

SEQ ID NO: 56 - protein, 1897 aa, Homo sapiens
MVPLVPALVMLGLVAGAHGDSKPVFIKVPEDQTGLSGGVASFVCQATGEPKPRITWMKKGKKVSSQRFEVIEFDDGAGSVLRIQPLRVQRDEAIYECTATNSLGEINTSAKLSVLEEEQLPPGFPSIDMGPQLKVVEKARTATMLCAAGGNPDPEISWFKDFLPVDPATSNGRIKQLRSGALQIESSEESDQGKYECVATNSAGTRYSAPANLYVRVRRVAPRFSIPPSSQEVMPGGSVNLTCVAVGAPMPYVKWMMGAEELTKEDEMPVGRNVLELSNVVRSANYTCVAISSLGMIEATAQVTVKALPKPPIDLVVTETTATSVTLTWDSGNSEPVTYYGIQYRAAGTEGPFQEVDGVATTRYSIGGLSPFSEYAFRVLAVNSIGRGPPSEAVRARTGEQAPSSPPRRVQARMLSASTMLVQWEPPEEPNGLVRGYRVYYTPDSRRPPNAWHKHNTDAGLLTTVGSLLPGITYSLRVLAFTAVGDGPPSPTIQVKTQQGVPAQPADFQAEVESDTRIQLSWLLPPQERIIMYELVYWAAEDEDQQHKVTFDPTSSYTLEDLKPDTLYRFQLAARSDMGVGVFTPTIEARTAQSTPSAPPQKVMCVSMGSTTVRVSWVPPPADSRNGVITQYSVAHEAVDGEDRGRHVVDGISREHSSWDLVGLEKWTEYRVWVRAHTDVGPGPESSPVLVRTDEDVPSGPPRKVEVEPLNSTAVHVYWKLPVPSKQHGQIRGYQVTYVRLENGEPRGLPIIQDVMLAEAQWRPEESEDYETTISGLTPETTYSVTVAAYTTKGDGARSKPKIVTTTGAVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGLKDSLLAHSSDPVEMRRLNYQTPGMRDHPPIPITDLADNIERLKANDGLKFSQEYESIDPGQQFTWENSNLEVNKPKNRYANVIAYDHSRVILTSIDGVPGSDYINANYIDGYRKQNAYIATQGPLPETMGDFWRMVWEQRTATVVMMTRLEEKSRVKCDQYWPARGTETCGLIQVTLLDTVELATYTVRTFALHKSGSSEKRELRQFQFMAWPDHGVPEYPTPILAFLRRVKACNPLDAGPMVVHCSAGVGRTGCFIVIDAMLERMKHEKTVDIYGHVTCMRSQRNYMVQTEDQYVFIHEALLEAATCGHTEVPARNLYAHIQKLGQVPPGESVTAMELEFKLLASSKAHTSRFISANLPCNKFKNRLVNIMPYELTRVCLQPIRGVEGSDYINASFLDGYRQQKAYIATQGPLAESTEDFWRMLWEHNSTIIVMLTKLREMGREKCHQYWPAERSARYQYFVVDPMAEYNMPQYILREFKVTDARDGQSRTIRQFQFTDWPEQGVPKTGEGFIDFIGQVHKTKEQFGQDGPITVHCSAGVGRTGVFITLSIVLERMRYEGVVDMFQTVKTLRTQRPAMVQTEDQYQLCYRAALEYLGSFDHYAT

SEQ ID NO: 57 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 58 - protein, 860 aa, Homo sapiens [sequence omitted]

SEQ ID NO: 59 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 60 - protein, 1250 aa, Homo sapiens
MARGDAGRGRGLLALTFCLLAARGELLLPQETTVELSCGVGPLQVILGPEQAAVLNCSLGAAAAGPPTRVTWSKDGDTLLEHDHLHLLPNGSLWLSQPLAPNGSDESVPEAVGVIEGNYSCLAHGPLGVLASQTAVVKLATLADFSLHPESQTVEENGTARFECHIEGLPAPIITWEKDQVTLPEEPRLIVLPNGVLQILDVQESDAGPYRCVATNSARQHFSQEALLSVAHRGSLASTRGQDVVIVAAPENTTVVSGQSVVMECVASADPTPFVSWVRQDGKPISTDVIVLGRTNLLIANAQPWHSGVYVCRANKPRTRDFATAAAELRVLAAPAITQAPEALSRTRASTARFVCRASGEPRPALRWLHNGAPLRPNGRVKVQGGGGSLVITQIGLQDAGYYQCVAENSAGMACAAASLAVVVREGLPSAPTRVTATPLSSSAVLVAWERPEMHSEQIIGFSLHYQKARGMDNVEYQFAVNNDTTELQVRDLEPNTDYEFYVVAYSQLGASRTSTPALVHTLDDVPSAAPQLSLSSPNPSDIRVAWLPLPPSLSNGQVVKYKIEYGLGKEDQIFSTEVRGNETQLMLNSLQPNKVYRVRISAGTAAGFGAPSQWMHHRTPSMHNQSHVPFAPAELKVQAKMESLVVSWQPPPHPTQISGYKLYWREVGAEEEANGDRLPGGRGDQAWDVGPVRLKKKVKQYELTQLVPGRLYEVKLVAFNKHEDGYAAVWKGKTEKAPAPDMPIQRGPPLPPAHVHAESNSSTSIWLRWKKPDFTTVKIVNYTVRFSPWGLRNASLVTYYTSSGEDILIGGLKPFTKYEFAVQSHGVDMDGPFGSVVERSTLPDRPSTPPSDLRLSPLTPSTVRLHWCPPTEPNGEIVEYLILYSSNHTQPEHQWTLLTTQGNIFSAEVHGLESDTRYFFKMGARTEVGPGPFSRLQDVITLQEKLSDSLDMHSVTGIIVGVCLGLLCLLACMCAGLRRSPHRESLPGLSSTATPGNPALYSRARLGPPSPPAAHELESLVHPHPQDWSPPPSDVEDRAEVHSLMGGGVSEGRSHSKRKISWAQPSGLSWAGSWAGCELPQAGPRPALTRALLPPAGTGQTLLLQALVYDAIKGNGRKKSPPACRNQVEAEVIVHSDFSASNGNPDLHLQDLEPEDPLPPEAPDLISGVGDPGQGAAWLDRELGGCELAAPGPDRLTCLPEAASASCSYPDLQPGEVLEETPGDSCQLKSPCPLGASPGLPRSPVSSSA

SEQ ID NO: 61 - protein, 653 aa, Homo sapiens
MPVGGLLPLFSSPAGGVLGGGLGGGGGRKGSGPAALRLTEKFVLLLVFSAFITLCFGAIFFLPDSSKLLSGVLFHSSPALQPAADHKPGPGARAEDAAEGRARRREEGAPGDPEAALEDNLARIRENHERALREAKETLQKLPEEIQRDILLEKKKVAQDQLRDKAPFRGLPPVDFVPPIGVESREPADAAIREKRAKIKEMMKHAWNNYKGYAWGLNELKPISKGGHSSSLFGNIKGATIVDALDTLFIMEMKHEFEEAKSWVEENLDFNVNAEISVFEVNIRFVGGLLSAYYLSGEEIFRKKAVELGVKLLPAFHTPSGIPWALLNMKSGIGRNWPWASGGSSILAEFGTLHLEFMHLSHLSGNPIFAEKVMNIRTVLNKLEKPQGLYPNYLNPSSGQWGQHHVSVGGLGDSFYEYLLKAWLMSDKTDLEAKKMYFDAVQAIETHLIRKSSSGLTYIAEWKGGLLEHKMGHLTCFAGGMFALGADAAPEGMAQHYLELGAEIARTCHESYNRTFMKLGPEAFRFDGGVEAIATRQNEKYYILRPEVMETYMYMWRLTHDPKYRKWAWEAVEALENHCRVNGGYSGLRDVYLLHESYDDVQQSFFLAETLKYLYLIFSDDDLLPLEHWIFNSEAHLLPILPKDKKEVEIREE

SEQ ID NO: 62 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 63 - protein, 543 aa, Homo sapiens
MEQVNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFMNPFNMPNLYQKLESDPRTRTLLSDPTYRELIEQLRNKPSDLGTKLQDPRIMTTLSVLLGVDLGSMDEEEEIATPPPPPPPKKETKPEPMEEDLPENKKQALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERLAYINPDLALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDSPEDVKRRAMADPEVQQIMSDPAMRLILEQMQKDPQALSEHLKNPVIAQKIQKLMDVGLIAIR

SEQ ID NO: 64 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 65 - protein, 1231 aa, Homo Sapiens
MRLLAKIICLMLWAICVAEDCNELPPRRNTEILTGSWSDQTYPEGGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRLKHRTGDELTYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKGGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

(19) United States
(12) Patent Application Publication
(10) Pub. No.: US 2010/0190652 A1
(43) Pub. Date: Jul. 29, 2010

(54) Biomarkers for Detection of Neonatal Sepsis in Biological Fluid

(76) Inventors: Srinivasa Nagalla, Hillsboro, OR (US); Michael Gravett, Seattle, WA (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/693,387

(22) Filed: Jan. 25, 2010

Related U.S. Application Data
(60) Provisional application No. 61/147,635, filed on Jan. 27, 2009.

(51) Int. Cl. C40B 30/00; G01N 33/53; G01N 33/573

(52) U.S. Cl. 506/7; 435/7.1; 435/7.4

(57) ABSTRACT

The present invention concerns the identification and detection of biological fluid biomarkers of neonatal sepsis using global proteomic approaches.

### RELATED APPLICATION

This application claims priority under 35 U.S.C. §119(e) to U.S. provisional application No. 61/147,635, filed Jan. 27, 2009, the entire contents of which are incorporated herein by reference.

### BACKGROUND OF THE INVENTION

1. Field of the Invention

The present invention concerns the identification and detection of biomarkers of neonatal sepsis and neonatal sepsis associated complications in biological fluids using global proteomic approaches.

2. Sequence Listing

The instant application contains a Sequence Listing which has been submitted via EFS-Web and is hereby incorporated by reference in its entirety. Said ASCII copy, created on Dec. 9, 2009, is named PTX-0013PR.txt, and is 412,016 bytes in size.

3. Description of the Related Art

Sepsis is a serious problem for neonates who are admitted for neonatal intensive care. It is associated with an increase in mortality, morbidity, and prolonged length of hospital stay. Thus, both the human and fiscal costs of these infections are high. Possible (“rule-out” or “suspected”) early onset septicemia remains the most common admitting diagnosis to the neonatal intensive care unit (NICU). Although the rate of early-onset sepsis increases with the degree of both prematurity and low birth weight, no specific laboratory test has been shown to be sufficiently precise to allow the identification of patients who have a “real” blood-stream infection and, therefore, who need to be treated with a full course of antibiotics. As a result, antibiotic use is many times the rate of “proven” sepsis and overuse of these agents facilitates the growth of resistant organisms in the neonatal intensive care unit. (Clarke 2004). In addition, the prolongation of hospital stay adds immeasurably to the cost of care in the NICU and enhances the risk of nosocomial septicemia from subsequent hospital acquired micro-organisms.

The U.S. Department of Health and Human Services Centers for Disease Control and Prevention defines early-onset infection as an infection during hospitalization that occurs during the first 72 hours of life, whereas late-onset infection occurs after that period of time. (Lopez 2002). Nosocomial infection is equivalent to late-onset, or infection after the first 72 hours of life. (Craft 2001). Infection rates may be stated as a percent of admissions, percent of liveborn infants, or by the number of infections per 1000 patient days. Early onset infection rates consistently run at approximately 2 per thousand live births. As 20% to 30% of preterm neonates may have two or more nosocomial infection episodes, infection rates per patient days probably gives a more accurate idea of magnitude in late-onset infection, whereas rates per patient group (admissions, liveborn infants, birth-weight range, gestational age range) give a good idea of attack or incidence rates.

The neonatal intensive care unit (NICU) nosocomial or late onset infection rate has increased over the past decade. (Craft 2001, Zafar 2001). The total number of neonates who develop nosocomial infection per admission varies from 6.2% (Ferguson 1996) to 33% (Hentschel 1999) or, when reported as total infections per 1000 patient days, the rate varies from 4.8 (Ferguson 1996) to 22 (Drews 1995). Blood-stream infections (nosocomial sepsis) vary from 3% to 28% of admissions. (Ferguson 1996, Hentschel 1999, Berger 1998, Horbar 2001, Nagata 2002). The variability of infection rates depends on the gestational age, the distribution of the infants surveyed for the report, and on the specific environment and care practices. (Gaynes 1996).

The gold standard for the diagnosis of true early-onset sepsis remains the finding of a positive blood culture for a known pathogen. Commonly, early onset sepsis will be considered present when a neonate has at least two of the following features in the clinical course, and a positive blood culture of 1 mL or greater volume:

1) Maternal history of fever >100.4° F., prolonged premature rupture of membranes during labor (>12 hours duration), or presumed chorioamnionitis

2) Malodorous or purulent appearing amniotic fluid at delivery

3) Clinical findings consistent with sepsis that may include any of the following signs: low 5 minute Apgar score (<6), pallor, cyanosis, hypotension, tachypnea, tachycardia, apnea, abdominal distension, poor feeding, or lethargy

4) Supporting laboratory data that includes a WBC count on CBC<8000/mm3 or >35,000/mm3; I:T neutrophil count >2; CRP >8; or pneumonia on chest radiograph.

This points to the need for the identification of sepsis-associated biomarkers within biological fluid obtained at delivery able to identify subjects with early-onset neonatal sepsis and neonatal sepsis associated complications to facilitate early treatment. Reductions in the risk of neonatal sepsis and its associated morbidities may well depend upon earlier identification of patients at risk.

### SUMMARY OF THE INVENTION

In one aspect, the invention provides a method for diagnosis of neonatal sepsis in a mammalian subject comprising: (a) testing in a sample of biological fluid obtained from said subject the level of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), relative to the level in normal biological fluid or biological fluid known to be indicative of neonatal sepsis; and (b)

diagnosing said subject with neonatal sepsis if said level shows a statistically significant difference relative to the level in said normal biological fluid, or does not show a statistically significant difference relative to the level in said biological fluid known to be indicative of neonatal sepsis.

In certain embodiments, the method includes testing the level of at least two, at least three, at least four, at least five, at least six, at least seven, and so on, of the listed proteins, in any combination.

In a specific embodiment, the subject is a human patient.

In certain embodiments, the biological fluid is selected from the group consisting of cord blood, cerebrospinal fluid, and neonatal serum. In a specific embodiment, the biological fluid is cord blood.

In another embodiment, the diagnosis is determined within 24 hours of birth.

In one embodiment, the testing is implemented using an apparatus adapted to determine the level of said proteins. In another embodiment, the testing is performed by using a software program executed by a suitable processor. In certain embodiments, the program is embodied in software stored on a tangible medium. In certain other embodiments, the tangible medium is selected from the group consisting of a CD-ROM, a floppy disk, a hard drive, a DVD, and a memory associated with the processor.

In certain embodiments, the methods of the invention further include a step of preparing a report recording the results of the testing or the diagnosis. In one embodiment, the report is recorded or stored on a tangible medium. In a specific embodiment, the tangible medium is paper. In another embodiment, the tangible medium is selected from the group consisting of a CD-ROM, a floppy disk, a hard drive, a DVD, and a memory associated with the processor.

In certain other embodiments, the methods of the invention further include a step of communicating the results of said diagnosis to an interested party. In one embodiment, the interested party is the patient or the attending physician. In another embodiment, the communication is in writing, by email, or by telephone.

In one embodiment, the method includes testing the level of proteins Insulin-like growth factor-binding protein 1 precursor (SEQ ID NO:36), Interleukin-6 precursor (SEQ ID NO:3), C-reactive protein precursor (SEQ ID NO:1), Beta-2-microglobulin precursor (SEQ ID NO:7), Cathepsin B precursor (SEQ ID NO:38), Cystatin-M precursor (SEQ ID NO:42), Insulin-like growth factor-binding protein 2 precursor (SEQ ID NO:44), Matrix metalloproteinase-9 (SEQ ID NO:64), Metalloproteinase inhibitor 1 precursor (SEQ ID NO:31), and Alpha-1-acid glycoprotein 1 (SEQ ID NO:65), and diagnosing said subject with neonatal sepsis, if one or more of said tested proteins shows a significant difference in the cord blood sample relative to normal cord blood. In a certain embodiment, the method includes diagnosing said subject with neonatal sepsis, if all of said tested proteins show a significant difference in the cord blood sample relative to normal cord blood.

In one embodiment, the method includes testing the level of proteins Insulin-like growth factor-binding protein 1 precursor (SEQ ID NO:36) and Interleukin-6 precursor (SEQ ID NO:3), and diagnosing said subject with neonatal sepsis, if one or more of said tested proteins shows a significant difference in the cord blood sample relative to normal cord blood. In other embodiments, the method includes testing the level of proteins Insulin-like growth factor-binding protein 1 precursor (SEQ ID NO:36) and C-reactive protein precursor (SEQ ID NO:1). In yet other embodiments, the method includes testing the level of proteins Insulin-like growth factor-binding protein 1 precursor (SEQ ID NO:36) and Beta-2-microglobulin precursor (SEQ ID NO:7). In still other embodiments, the method includes testing the level of proteins Insulin-like growth factor-binding protein 1 precursor (SEQ ID NO:36) and Cathepsin B precursor (SEQ ID NO:38). In still other embodiments, the method includes testing the level of proteins Insulin-like growth factor-binding protein 1 precursor (SEQ ID NO:36) and Cystatin-M precursor (SEQ ID NO:42). In still other embodiments, the method includes testing the level of proteins Insulin-like growth factor-binding protein 1 precursor (SEQ ID NO:36) and Insulin-like growth factor-binding protein 2 precursor (SEQ ID NO:44). In still other embodiments, the method includes testing the level of proteins Insulin-like growth factor-binding protein 1 precursor (SEQ ID NO:36) and Matrix metalloproteinase-9 (SEQ ID NO:64). In still other embodiments, the method includes testing the level of proteins Insulin-like growth factor-binding protein 1 precursor (SEQ ID NO:36) and Metalloproteinase inhibitor 1 precursor (SEQ ID NO:31). In still other embodiments, the method includes testing the level of proteins Insulin-like growth factor-binding protein 1 precursor (SEQ ID NO:36) and Alpha-1-acid glycoprotein 1 (SEQ ID NO:65).

In certain embodiments, the level of the listed proteins is determined by an immunoassay, by mass spectrometry, or by using a protein array.

In another aspect, the invention provides the use of any one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), in the manufacture of a proteomic profile of a biological fluid for the early diagnosis of neonatal sepsis in a subject.

In certain embodiments, the proteomic profile comprises information of the level of at least two of said proteins, at least three, at least four, at least five, at least six, at least seven, and so on, of the listed proteins, in any combination.

In a specific embodiment, the subject is a human patient.

In certain embodiments, the biological fluid is selected from the group consisting of cord blood, neonatal serum and cerebrospinal fluid. In a specific embodiment, the biological fluid is cord blood.

In one embodiment, the proteomic profile comprises information of the level of said proteins and wherein the diagnosis of said subject with neonatal sepsis is made if one or more of said tested proteins shows a significant difference in the biological fluid sample relative to normal biological fluid.

In another embodiment, the diagnosis is determined within 24 hours of birth.

In one embodiment, the proteomic profile comprises information of the level of proteins Insulin-like growth factor-binding protein 1 precursor (SEQ ID NO:36), Interleukin-6 precursor (SEQ ID NO:3), C-reactive protein precursor (SEQ ID NO:1), Beta-2-microglobulin precursor (SEQ ID NO:7), Cathepsin B precursor (SEQ ID NO:38), Cystatin-M precursor (SEQ ID NO:42), Insulin-like growth factor-binding protein 2 precursor (SEQ ID NO:44), Matrix metalloproteinase-9 (SEQ ID NO:64), Metalloproteinase inhibitor 1 precursor (SEQ ID NO:31), and Alpha-1-acid glycoprotein 1 (SEQ ID NO:65), and wherein the diagnosis of said subject with neonatal sepsis is made if one or more of said tested proteins shows a significant difference in the biological fluid sample relative to normal biological fluid. In a specific embodiment, the diagnosis of said subject with neonatal sepsis is made if all of said tested proteins show a significant difference in the biological fluid sample relative to normal biological fluid.

In certain embodiments, the level of the listed proteins is determined by an immunoassay, by mass spectrometry, or by using a protein array.

In yet another aspect, the invention provides an immunoassay kit comprising antibodies and reagents for the detection of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63).

In another aspect, the invention provides an immunoassay kit comprising antibodies and reagents for the detection of one or more proteins selected from the group consisting of Insulin-like growth factor-binding protein 1 precursor (SEQ ID NO:36), Interleukin-6 precursor (SEQ ID NO:3), C-reactive protein precursor (SEQ ID NO:1), Beta-2-microglobulin precursor (SEQ ID NO:7), Cathepsin B precursor (SEQ ID NO:38), Cystatin-M precursor (SEQ ID NO:42), Insulin-like growth factor-binding protein 2 precursor (SEQ ID NO:44), Matrix metalloproteinase-9 (SEQ ID NO:64), Metalloproteinase inhibitor 1 precursor (SEQ ID NO:31), and Alpha-1-acid glycoprotein 1 (SEQ ID NO:65).

In one embodiment, the immunoassay kit includes antibodies and reagents for the detection of all of listed proteins.

In yet another aspect, the invention provides a report comprising the results of and/or diagnosis based on a test comprising (a) testing in a sample of biological fluid obtained from said subject the level of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), relative to the level in normal biological fluid or biological fluid known to be indicative of neonatal sepsis; and (b) diagnosing said subject with neonatal sepsis if said level shows a statistically significant difference relative to the level in said normal biological fluid, or does not show a statistically significant difference relative to the level in said biological fluid known to be indicative of neonatal sepsis.

In still another aspect, the invention provides a tangible medium storing the results of and/or diagnosis based on a test comprising (a) testing in a sample of biological fluid obtained from said subject the level of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), relative to the level in normal biological fluid or biological fluid known to be indicative of neonatal sepsis; and (b) diagnosing said subject with neonatal sepsis if said level shows a statistically significant difference relative to the level in said normal biological fluid, or does not show a statistically significant difference relative to the level in said biological fluid known to be indicative of neonatal sepsis.

### BRIEF DESCRIPTION OF THE DRAWINGS

FIG. 1 depicts Cord Blood DIGE Analysis: (A) control (red) vs. suspected sepsis (SS) (green) DIGE gel. (B) control (red) vs. confirmed sepsis (CS) (green) DIGE gel. Spots that are not differentially expressed appear yellow. (C) Differentially expressed spots between suspected sepsis (SS) vs. control. (D) Differentially expressed spots between confirmed sepsis (CS) vs. control. Spots highlighted in red were determined to be ≧2 fold down regulated and spots highlighted in green were determined to be ≧2 fold up regulated.

FIG. 2 depicts spectral counts of cord blood proteins from control, suspected sepsis (SS), and confirmed sepsis (CS) neonatal subjects are loaded into GeneMaths software for differential expression visualization. Proteins are hierarchically clustered using Euclidean distance learning in 200 iterations and shown in FIG. 2A. Selected sub clusters of up regulated (FIG. 2B) and down regulated proteins (FIG. 2C) are also shown. Positions of the selected sub clusters in FIG. 2A are marked accordingly.

### DETAILED DESCRIPTION OF THE PREFERRED EMBODIMENT

### I. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994) provides one skilled in the art with a general guide to many of the terms used in the present application.

The term “neonatal sepsis” is used herein to describe infection of the blood of a newborn and includes all complications associated with such infection. Neonatal sepsis associated complications include but are not limited to respiratory distress syndrome (RDS), central nervous system (CNS) complications, e.g., periventricular hemorrhage and periventricular leukomalacia, mental retardation, cerebral palsy (CP), disability and death.

The term “proteome” is used herein to describe a significant portion of proteins in a biological sample at a given time. The concept of proteome is fundamentally different from the genome. While the genome is virtually static, the proteome continually changes in response to internal and external events.

The term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins are also included. Thus the proteomic profile may, for example, be based on differences in the electrophoretic properties of proteins, as determined by two-dimensional gel electrophoresis, e.g. by 2-D PAGE, and can be represented, e.g. as a plurality of spots in a two-dimensional electrophoresis gel.

Differential expression profiles may have important diagnostic value, even in the absence of specifically identified proteins. Single protein spots can then be detected, for example, by immunoblotting, multiple spots or proteins using protein microarrays. The proteomic profile typically represents or contains information that could range from a few peaks to a complex profile representing 50 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50, or at least 60, or at least 65, or at least 70, or at least 75, or at least 80, or at least 85, or at least 85, or at least 90, or at least 95, or at least 100, or at least 125, or at least 150, or at least 175, or at least 200 proteins.

The term “biological fluid” as used herein refers to refers to liquid material derived from a human or other animal. Biological fluids include, but are not limited to, cord blood, neonatal serum, cerebrospinal fluid (CSF), cervical-vaginal fluid (CVF), amniotic fluid, serum, plasma, urine, cerebrospinal fluid, breast milk, mucus, saliva, and sweat.

“Patient response” can be assessed using any endpoint indicating a benefit to the patient, including, without limitation, (1) inhibition, at least to some extent, of the progression of a pathologic condition, (2) prevention of the pathologic condition, (3) relief, at least to some extent, of one or more symptoms associated with the pathologic condition; (4) increase in the length of survival following treatment; and/or (5) decreased mortality at a given point of time following treatment.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The designation of any particular protein, as used herein, includes all fragments, precursors, and naturally occurring variants, such as alternatively spliced and allelic variants and isoforms, as well as soluble forms of the protein named, along with native sequence homologs (including all naturally occurring variants) in other species. Thus, for example, when it is stated that the level of haptoglobin precursor (Swiss-Prot Acc. No. P00738) is tested, the statement specifically includes testing any fragments, precursers, or naturally occurring variant of the protein listed under Swiss-Prot Acc. No. P00738, as well as its non-human homologs and naturally occurring variants thereof, if subject is non-human.

### II. Detailed Description

The present invention concerns methods and means for an early, reliable and non-invasive testing of neonatal sepsis and/or neonatal sepsis associated complications by proteomic analysis of biological fluid, such as cord blood. The invention further concerns identification of biomarkers of neonatal sepsis. In another aspect, the invention concerns the use of proteins in the preparation or manufacture of proteomic profiles as a means for the early determination of neonatal sepsis. The invention utilizes proteomics techniques well known in the art, as described, for example, in the following textbooks, the contents of which are hereby expressly incorporated by reference: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research: Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

One skilled in the art will recognize many methods and materials similar or equivalent to those described herein, which could be used in the practice of the present invention. Indeed, the present invention is in no way limited to the methods and materials described.

1. Identification of Proteins and Polypeptides Expressed in Biological Fluids

According to the present invention, proteomics analysis of biological fluids can be performed using a variety of methods known in the art. Biological fluids include, for example, cord blood, neonatal serum, cerebrospinal fluid (CSF), cervical-vaginal fluid (CVF), amniotic fluid, serum, plasma, urine, cerebrospinal fluid, breast milk, mucus, saliva, and sweat.

Typically, protein patterns (proteome maps) of samples from different sources, such as normal biological fluid (normal sample) and a test biological fluid (test sample), are compared to detect proteins that are up- or down-regulated in a disease. These proteins can then be excised for identification and full characterization, e.g. using peptide-mass fingerprinting and/or mass spectrometry and sequencing methods, or the normal and/or disease-specific proteome map can be used directly for the diagnosis of the disease of interest, or to confirm the presence or absence of the disease.

In comparative analysis, it is important to treat the normal and test samples exactly the same way, in order to correctly represent the relative level or abundance of proteins, and obtain accurate results. The required amount of total proteins will depend on the analytical technique used, and can be readily determined by one skilled in the art. The proteins present in the biological samples are typically separated by two-dimensional gel electrophoresis (2-DE) according to their pI and molecular weight. The proteins are first separated by their charge using isoelectric focusing (one-dimensional gel electrophoresis). This step can, for example, be carried out using immobilized pH-gradient (IPG) strips, which are commercially available. The second dimension is a normal SDS-PAGE analysis, where the focused IPG strip is used as the sample. After 2-DE separation, proteins can be visualized with conventional dyes, like Coomassie Blue or silver staining, and imaged using known techniques and equipment, such as, e.g. Bio-Rad GS800 densitometer and PDQUEST software, both of which are commercially available. Individual spots are then cut from the gel, destained, and subjected to tryptic digestion. The peptide mixtures can be analyzed by mass spectrometry (MS). Alternatively, the peptides can be separated, for example by capillary high pressure liquid chromatography (HPLC) and can be analyzed by MS either individually, or in pools.

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. Mass spectrometry has been widely used in protein analysis, especially since the invention of matrix-assisted laser-desorption ionisation/time-of-flight (MALDI-TOF) and electrospray ionisation (ESI) methods. There are several versions of mass analyzer, including, for example, MALDI-TOF and triple or quadrupole-TOF, or ion trap mass analyzer coupled to ESI. Thus, for example, a Q-Tof-2 mass spectrometer utilizes an orthogonal time-of-flight analyzer that allows the simultaneous detection of ions across the full mass spectrum range. For further details see, e.g. Chemusevich et al., J. Mass Spectrom. 36:849-865 (2001). If desired, the amino acid sequences of the peptide fragments and eventually the proteins from which they derived can be determined by techniques known in the art, such as certain variations of mass spectrometry, or Edman degradation.

2. Early Detection of Neonatal Sepsis

Neonatal sepsis, defined as infection of the blood of a newborn, is difficult to diagnose clinically. Despite advances in neonatal care, the mortality and morbidity from neonatal sepsis remains high (Stoll 2002). Neonatal sepsis is an important contributor to neonatal morbidity including poor neurodevelopmental outcomes and neonatal death. Neonatal sepsis associated complications include, for example, respiratory distress syndrome (RDS), central nervous system (CNS) complications, cerebral palsy (CP), disability and death.

The highest rates of neonatal sepsis occur in low-birth-weight (LBW) infants, those with depressed respiratory function at birth, and those with maternal perinatal risk factors. Risk factors for early-onset neonatal sepsis include obstetric complications, including preterm delivery, premature rupture of membranes, maternal bleeding, e.g., as caused by placenta previa, abruptio placentae, infection of the amniotic fluid, placenta, urinary tract or endometrium, toxemia, precipitous delivery, and frequent vaginal examinations during delivery. Extended hospital stays and contaminated hospital equipment are common causes of late-onset neonatal sepsis. Organisms which can cause neonatal sepsis include the following non-limiting examples: Coagulase-negative staphylococci, including S. epidermidis, S. haemolyticus, S. hominis, S. warneri, S. saprophyticus, S. cohnii, and S. capitis, Group B Streptococcus, Staphylococcus aureus, Enterococcus fecalis and E. faecium, Listeria monocytogenes, Escherichia coli, P. aeruginosa, Haemophilus influenzae, Streptococcus bovis, α-hemolytic streptococci, Streptococcus pneumoniae, Neisseria meningitides and N. gonorrhoeae. Typically, the organisms which give rise to early-onset neonatal sepsis are acquired intrapartum as an ascending infection from the cervix, transplacentally from the mother or as the fetus passes through the birth canal.

Unfortunately, due to nonspecific and subtle early signs, the diagnosis of neonatal sepsis is difficult. Signs and symptoms of neonatal sepsis include, for example, body temperature changes breathing problems, diarrhea, low blood sugar, reduced movements, reduced sucking, seizures, slow heart rate, swollen belly area, vomiting, and jaundice. The gold standard for diagnosing neonatal sepsis is blood culture; however, negative blood cultures occur even when strong clinical indicators of septicemia are present and even in cases where bacterial infection is later proven by autopsy (Kaufman D, Fairchild K D, Clin Microbiol Rev. 2004 July; 17(3):638-80). Furthermore, it is often difficult to obtain a sufficient blood sample in neonates, particularly preterm neonates. Given its rapid progression and high mortality rate, rapid empiric antibiotic therapy is typically administered, pending blood culture results. Initial therapy can include ampicillin or penicillin G and an aminoglycoside, e.g., gentamicin, or cefotaxime. Given negative outcomes associated with neonatal sepsis and the lack of confidence in currently available means for detecting neonatal sepsis, use of antibiotic treatment is not only common but prolonged, which contributes to drug resistance among neonatal pathogens. Therefore, development of early, reliable and non-invasive markers for neonatal sepsis and neonatal sepsis associated complications is imperative to allow for therapy and intervention to optimize the outcome for the neonate and to minimize the use or prolonged use of potentially unnecessary antibiotics.

3. Early Detection and Diagnosis of Neonatal Sepsis Using Biomarkers in Biological Fluids

In one aspect, the present invention provides reliable, non-invasive methods for the diagnosis of the neonatal sepsis and neonatal sepsis associated complications using biomarkers identified in biological fluid, such as cord blood, using a proteomics approach. In certain embodiment, the biomarkers associated with neonatal sepsis are predictors for early and late onset central nervous system (CNS) complications. In one embodiment, the biomarkers are predictors for periventricular hemorrhage and/or periventricular leukomalacia. In another embodiment, the biomarkers are predictors for mental retardation.

In one embodiment, the instant invention allows detection of neonatal sepsis and neonatal sepsis associated complications biomarkers within about 30 minutes and 24 hours of sample collection. In certain embodiments, early-onset neonatal sepsis and/or an associated complication is diagnosed within about 30 minutes and 48 hours of sample collection. In another embodiment, early-onset neonatal sepsis and/or an associated complication is diagnosed within about 48 hours of sample collection. In yet another embodiment, early-onset neonatal sepsis and/or an associated complication is diagnosed within about 24 hours of sample collection. In still another embodiment, early-onset neonatal sepsis and/or an associated complication is diagnosed within about 12 hours of sample collection. In another embodiment, early-onset neonatal sepsis and/or an associated complication is diagnosed within about 4 hours of sample collection. In other embodiments, early-onset neonatal sepsis and/or an associated complication is diagnosed within about 2 hours of sample collection. In one embodiment, early-onset neonatal sepsis and/or an associated complication is diagnosed within about 1 hours of sample collection. In another embodiment, early-onset neonatal sepsis and/or an associated complication is diagnosed within about 30 minutes of sample collection.

In certain other embodiments, early-onset neonatal sepsis and/or an associated complication is diagnosed within about 30 minutes and 48 hours of birth. In another embodiment, early-onset neonatal sepsis and/or an associated complication is diagnosed within about 48 hours of birth. In yet another embodiment, early-onset neonatal sepsis and/or an associated complication is diagnosed within about 24 hours of birth. In still another embodiment, early-onset neonatal sepsis and/or an associated complication is diagnosed within about 12 hours of birth. In another embodiment, early-onset neonatal sepsis and/or an associated complication is diagnosed within about 4 hours of birth. In other embodiments, early-onset neonatal sepsis and/or an associated complication is diagnosed within about 2 hours of birth. In one embodiment, early-onset neonatal sepsis and/or an associated complication is diagnosed within about 1 hours of birth. In another embodiment, early-onset neonatal sepsis and/or an associated complication is diagnosed within about 30 minutes of birth.

As noted before, in the context of the present invention the term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins are also included. Although it is possible to identify and sequence all or some of the proteins present in the proteome of a biological fluid, this is not necessary for the diagnostic use of the proteomic profiles generated in accordance with the present invention. Diagnosis of a particular disease can be based on characteristic differences (unique expression signatures) between a normal proteomic profile, and proteomic profile of the same biological fluid obtained under the same circumstances, when the disease or pathologic condition to be diagnosed is present. The unique expression signature can be any unique feature or motif within the proteomic profile of a test or reference biological sample that differs from the proteomic profile of a corresponding normal biological sample obtained from the same type of source, in a statistically significant manner. For example, if the proteomic profile is presented in the form of a mass spectrum, the unique expression signature is typically a peak or a combination of peaks that differ, qualitatively or quantitatively, from the mass spectrum of a corresponding normal sample. Thus, the appearance of a new peak or a combination of new peaks in the mass spectrum, or any statistically significant change in the amplitude or shape of an existing peak or combination of existing peaks, or the disappearance of an existing peak, in the mass spectrum can be considered a unique expression signature. When the proteomic profile of the test sample obtained from a mammalian subject is compared with the proteomic profile of a reference sample comprising a unique expression signature characteristic of a pathologic maternal or fetal condition, the mammalian subject is diagnosed with such pathologic condition if it shares the unique expression signature with the reference sample.

A particular pathologic maternal/fetal condition can be diagnosed by comparing the proteomic profile of a biological fluid obtained from the subject to be diagnosed with the proteomic profile of a normal biological fluid of the same kind, obtained and treated in the same manner. If the proteomic profile of the test sample is essentially the same as the proteomic profile of the normal sample, the subject is considered to be free of the subject pathologic maternal/fetal condition. If the proteomic profile of the test sample shows a unique expression signature relative to the proteomic profile of the normal sample, the subject is diagnosed with the maternal/fetal condition in question.

Alternatively or in addition, the proteomic profile of the test sample may be compared with the proteomic profile of a reference sample, obtained from a biological fluid of a subject independently diagnosed with the pathologic maternal/fetal condition ion question. In this case, the subject is diagnosed with the pathologic condition if the proteomic profile of the test sample shares at least one feature, or a combination of features representing a unique expression signature, with the proteomic profile of the reference sample.

Statistical methods for comparing proteomic profiles are well known in the art. For example, in the case of a mass spectrum, the proteomic profile is defined by the peak amplitude values at key mass/charge (M/Z) positions along the horizontal axis of the spectrum. Accordingly, a characteristic proteomic profile can, for example, be characterized by the pattern formed by the combination of spectral amplitudes at given M/Z vales. The presence or absence of a characteristic expression signature, or the substantial identity of two profiles can be determined by matching the proteomic profile (pattern) of a test sample with the proteomic profile (pattern) of a reference or normal sample, with an appropriate algorithm. A statistical method for analyzing proteomic patterns is disclosed, for example, in Petricoin III, et al., The Lancet 359:572-77 (2002); Issaq et al., Biochem Biophys Commun 292:587-92 (2002); Ball et al., Bioinformatics 18:395-404 (2002); and Li et al., Clinical Chemistry Journal, 48:1296-1304 (2002).

In a particular embodiment, the diagnostic tests of the present invention are performed in the form of protein arrays or immunoassays.

4. Protein Arrays

In recent years, protein arrays have gained wide recognition as a powerful means to detect proteins, monitor their expression levels, and investigate protein interactions and functions. They enable high-throughput protein analysis, when large numbers of determinations can be performed simultaneously, using automated means. In the microarray or chip format, that was originally developed for DNA arrays, such determinations can be carried out with minimum use of materials while generating large amounts of data.

Although proteome analysis by 2D gel electrophoresis and mass spectrometry, as described above, is very effective, it does not always provide the needed high sensitivity and this might miss many proteins that are expressed at low abundance. Protein microarrays, in addition to their high efficiency, provide improved sensitivity. Protein arrays are formed by immobilizing proteins on a solid surface, such as glass, silicon, micro-wells, nitrocellulose, PVDF membranes, and microbeads, using a variety of covalent and non-covalent attachment chemistries well known in the art. The solid support should be chemically stable before and after the coupling procedure, allow good spot morphology, display minimal nonspecific binding, should not contribute a background in detection systems, and should be compatible with different detection systems.

In general, protein microarrays use the same detection methods commonly used for the reading of DNA arrays. Similarly, the same instrumentation as used for reading DNA microarrays is applicable to protein arrays.

Thus, capture arrays (e.g. antibody arrays) can be probed with fluorescently labeled proteins from two different sources, such as normal and diseased biological fluids. In this case, the readout is based on the change in the fluorescent signal as a reflection of changes in the expression level of a target protein. Alternative readouts include, without limitation, fluorescence resonance energy transfer, surface plasmon resonance, rolling circle DNA amplification, mass spectrometry, resonance light scattering, and atomic force microscopy.

For further details, see, for example, Zhou H, et al., Trends Biotechnol. 19:S34-9 (2001); Zhu et al., Current Opin. Chem. Biol. 5:40-45-(2001); Wilson and Nock, Angew Chem Int Ed Engl 42:494-500 (2003); and Schweitzer and Kingsmore, Curr Opin Biotechnol 13:14-9 (2002). Biomolecule arrays are also disclosed in U.S. Pat. No. 6,406,921, issued Jun. 18, 2002, the entire disclosure of which is hereby expressly incorporated by reference.

5. Immunoassays

The diagnostic assays of the present invention can also be performed in the form of various immunoassay formats, which are well known in the art. There are two main types of immunoassays, homogenous and heterogeneous. In homogenous immunoassays, both the immunological reaction between an antigen and an antibody and the detection are carried out in a homogenous reaction. Heterogeneous immunoassays include at least one separation step, which allows the differentiation of reaction products from unreacted reagents.

ELISA is a heterogeneous immunoassay, which has been widely used in laboratory practice since the early 1970's. The assay can be used to detect antigensin various formats.

In the “sandwich” format the antigen being assayed is held between two different antibodies. In this method, a solid surface is first coated with a solid phase antibody. The test sample, containing the antigen (i.e. a diagnostic protein), or a composition containing the antigen, being measured, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. The amount of color intensity resulting from the enzyme-substrate interaction is a measure of the amount of antigen in the sample tested. Homogenous immunoassays include, for example, the Enzyme Multiplied Immunoassay Technique (EMIT), which typically includes a biological sample comprising the compound or compounds to be measured, enzyme-labeled molecules of the compound(s) to be measured, specific antibody or antibodies binding the compound(s) to be measured, and a specific enzyme chromogenic substrate. In a typical EMIT excess of specific antibodies is added to a biological sample. If the biological sample contains the proteins to be detected, such proteins bind to the antibodies. A measured amount of the corresponding enzyme-labeled proteins is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the Ag-enzyme complex is Ab-bound makes the EMIT a unique system, enabling the test to be performed without a separation of bound from unbound compounds as is necessary with other immunoassay methods.

Part of this invention is also an immunoassay kit. In one aspect, the invention includes a sandwich immunoassay kit comprising a capture antibody and a detector antibody. The capture antibody and detector antibody can be monoclonal or polyclonal. In another aspect, the invention includes a diagnostic kit comprising lateral flow devices, such as immunochromatographic strip (ICS) tests, using immunoflowchromatography. The lateral flow devices employ lateral flow assay techniques as generally described in U.S. Pat. Nos. 4,943,522; 4,861,711; 4,857,453; 4,855,240; 4,775,636; 4,703,017; 4,361, 537; 4,235,601; 4,168,146; 4,094,647, the entire contents of each of which is incorporated by reference. In yet another aspect, the immunoassay kit may comprise, for example, in separate containers (a) monoclonal antibodies having binding specificity for the polypeptides used in the diagnosis of a particular maternal/fetal condition, such as neonatal sepsis; (b) and anti-antibody immunoglobulins. This immunoassay kit may be utilized for the practice of the various methods provided herein. The monoclonal antibodies and the anti-antibody immunoglobulins may be provided in an amount of about 0.001 mg to about 100 grams, and more preferably about 0.01 mg to about 1 gram. The anti-antibody immunoglobulin may be a polyclonal immunoglobulin, protein A or protein G or functional fragments thereof, which may be labeled prior to use by methods known in the art. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like. The test kit may be packaged in any suitable manner, typically with all elements in a single container along with a sheet of printed instructions for carrying out the test.

6. Diagnostic and Treatment Methods

The diagnostic methods of the present invention are valuable tools for practicing physicians to make quick treatment decisions, which are often critical for the survival of the neonate. Thus, for example, if a neonate shows symptoms of neonatal sepsis, or is otherwise at risk for neonatal sepsis, it is important to take immediate steps to treat the condition and improve the chances of the survival of the neonate.

Following the measurement or obtainment of the expression levels of the proteins identified herein, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the expression level in a test subject of one or more of the biomarkers presented herein is communicated to the subject as soon as possible after the assay is completed and the diagnosis and/or prediction is generated. The one or more biomarkers identified and quantified in the methods described herein can be contained in one or more panels. The number of biomarkers comprising a panel can include 1 biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, 20 biomarkers, etc. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761, the entire contents of which are incorporated by reference herein; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

To facilitate diagnosis, the reference and/or subject biomarker profiles or expression level of one or more of the biomarkers presented herein of the present invention can be displayed on a display device, contained electronically, or in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

Further details of the invention will be apparent from the following non-limiting examples. All references cited throughout the disclosure, and the references cited therein, are expressly incorporated by reference herein.

### Example 1

### Identification of Cord Blood Biomarkers of Neonatal Sepsis Using Global Proteomic Approaches

### Experimental Methods

Sample Collection: Umbilical cord blood samples from a prospective observational cohort of 82 women in spontaneous preterm labor at 20-34 weeks' gestation were analyzed. Early-onset neonatal sepsis was defined as a positive neonatal blood culture within 72 hours of delivery. Of 82 subjects, 71 delivered at <34 weeks and 5 of neonates had confirmed neonatal sepsis (neonatal blood culture positive) and 8 of the neonates had diagnosis of suspected sepsis (blood culture negative, clinical symptoms suggestive of infection).

Immunodepletion of cord serum: Serum samples used for 2-DLC experiments were depleted of 12 most abundant proteins (albumin, IgG, IgA, IgM, α-1-anti-trypsin, transferrin, haptoglobin, α-1-acid glycoprotein, α-2-macroglobulin, fibrinogen, apolipoproteins A-I and A-II) using IgY-12 LC2 proteome partitioning system (Beckman Coulter, Fullerton, Calif.). Appropriate fractions were collected, concentrated, and buffer exchanged with 10 mM Tris (pH 8.4). Protein concentration was determined using a DC protein assay kit (Bio-Rad, Hercules, Calif.).

Differential Gel Electrophoresis (DIGE): Following protein assay, 50 μg of protein was labeled with CyDye DIGE Fluor minimal dye (GE Lifesciences) at a concentration of 400 pm of dye. Different dyes (Cy5, Cy3, Cy2) were used to label control, suspected sepsis (SS), or confirmed sepsis (CS) cord blood serum (CBS) samples, respectively. Labeled proteins were dissolved in IEF buffer containing 0.5% ampholytes and rehydrated on to a 24 cm IPG strip (pH 4-7) for 12 h at room temperature. After rehydration, the IPG strip was subjected to isoelectric focusing for ˜10 h to attain a total of 64000 volt*hours. Focused proteins in the IPG strip were first reduced by equilibrating with buffer containing 1% DTT for 15 min and then alkylated with buffer containing 2.5% IAA. After reduction and alkylation steps, the IPG strip was loaded on to a gradient (8˜16%) polyacrylamide gel (24×20 cm) and the SDS-PAGE was conducted at 85 V for 18 h to resolve proteins in the second dimension. After electrophoresis, the gel was scanned in a Typhoon 9400 scanner (GE Lifesciences) using appropriate lasers and filters with PMT voltage set at 600. Images in different channels were overlaid using selected colors and differences were visualized using ImageQuant TL software (v7.0, GE Lifesciences). Raw scanned image files were loaded into Phoretix 2D Evolution (Nonlinear Dynamics), and difference maps were generated for confirmed and suspected sepsis versus control.

2-DLC Sample Processing: Following protein assay, 1 mg portions of samples were digested with trypsin, and resulting peptides were separated with strong cation exchange (SCX) chromatography. Samples were dried and dissolved in 105 μl of digestion buffer containing 0.2 M NH4HCO3 and 0.3% Rapigest (Waters, Milford, Mass.) (pH 8.5). Cysteine residues were reduced and alkylated by incubating in 12.5 μL of 0.1 M DTT at 50° C. for 45 min followed by dark room incubation in 7 μL of 0.5 M iodoacetamide for another 30 min. Proteins were digested for 2 h at 37° C. by adding 4 μL of 0.1 M CaCl2 and sequencing grade trypsin (Trypsin Gold, Promega) at an enzyme to substrate ratio of 33:1. Digestion was stopped by adding 60 μL of 0.2 M HCl and resulting peptides were purified using C18 SepPak Plus cartridges (Waters, Milford, Mass.).

SCX chromatography was performed using a 100×2.1 mm polysulfoethyl A column (The Nest Group, Southborough, Mass.). Mobile phase A contained 10 mM potassium phosphate (pH 3) and 25% acetonitrile (ACN). Mobile phase B was identical except that it contained 350 mM KCl. Following loading and washing in mobile phase A, peptides were eluted using a linear gradient of 0-50% B over 45 min, followed by a linear gradient of 50-100% B over 15 min, followed by a 20 min wash at 100% A. A total of 95 one-minute fractions were collected, dried by vacuum centrifugation, and re-dissolved by shaking in 100 μL of 0.1% TFA. Peptide fractions were desalted using a 96-well spin column, Vydac C18 silica (The Nest Group, Southborough, Mass.). The desalted fractions were consolidated into 35 fractions, evaporated, and dissolved in 20 μL of 5% formic acid (FA) for LC-MS/MS analysis.

LC-MS/MS Analysis: Portions of each fraction were analyzed by LC/MS using an Agilent 1100 series capillary LC system and an LTQ ion trap mass spectrometer (Thermo Electron, San Jose, Calif., USA) with an Ion Max electrospray source fitted with a 34-gauge metal needle kit (ThermoFinnigan, San Jose, Calif.). Samples were applied at 20 μL/min to a trap cartridge, and then switched onto a 0.5×250 mm Zorbax SB-C18 column (Agilent Technologies, Palo Alto, Calif., USA) using mobile phase A containing 0.1% FA. Mass spectra files were generated from raw data using Bioworks Browser software (version 3.1, ThermoFinnigan, San Jose, Calif.). A total of 1,195,238 tandem mass spectra were generated from all LC-MS/MS analyses.

Peptide and Protein Identification: Tandem mass spectra were searched against a composite protein database containing forward and reversed entries (decoy proteins) of Swiss-Prot (version 54.2) database selected for human subspecies. All searches were performed using X! Tandem (Fenyo 2003) search engine configured to use a mass tolerance of 1.8 Da and 0.4 Da for parent and fragment ions, trypsin enzyme specificity, fixed carbamidomethyl modification on cysteine residues, and several potential in vivo and in vitro modifications. Peptide and protein identifications in all samples were compiled together to generate a comprehensive cord blood proteome, using probabilistic protein identification algorithms (Nesvizhskii 2003) implemented in Scaffold software (version 1.6, Proteome Software, Portland, Oreg.). Peptide identifications with probability ≧0.8 are considered as likely to be present in the sample. Protein identifications with at least two unique peptide identifications are considered to be present in cord blood.

Label-Free Quantification: The total number of tandem mass spectra matched to a protein (spectral counting) is a label-free, sensitive, and semi-quantitative measure for estimating its abundance in complex mixtures. (Liu 2004). The difference of a protein's spectral counts between two complex samples was used to quantify its relative expression. (Old 2005). In this study, cord blood proteins with at least two unique peptide identifications in one sample were considered for label-free quantification. Homologous proteins (sequence homology >50%) with shared spectral counts were combined into single entry. Shared spectral counts of non-homologous were assigned to the protein with highest number of spectral matches (Occam's razor). Spectral counts of curated proteins were subjected to independent pair-wise comparisons between control and CS neonatal subjects were used to quantify the relative expression of a protein. (Gravett 2007, Nagalla 2007, Pereira 2007, Zybailov 2006). Proteins with a p-value of ≦0.05 in the pair-wise comparison were considered as significantly differentially expressed between the samples. The fold expression change (FC) of differentially expressed proteins was quantified using the equation described in Old et al. 2005).

Enzyme Linked Immunosorbent Assay (ELISA): 10 candidate biomarkers for detection of sepsis were measured with solid phase sandwich immunoassays. Available commercial antibodies and antigens were purchased from various vendors to prepare immunoassays. Standard curves were developed using known quantities of recombinant proteins or standards provided by manufacturer, to reference sample concentrations. All assays were performed in triplicate and interassay and intrasaay coefficient of variations ranged from 3-7%.

One-way analyses of variance (ANOVA) were conducted to compare log-transformed ELISA values of samples from subjects without sepsis and subjects with confirmed sepsis. For presentation, we transformed the average log value back to original units (harmonic mean), and applied the Bonferroni correction to account for multiple comparisons. Based on results from individual protein comparisons, we evaluated the classification performance of several different combinations of 2, 3 or 4 proteins using logistic regression models. Receiver operating characteristic (ROC) curves were computed based on the risk scores from each of the multi-protein models. Descriptive and inferential statistics were computed using SAS software (v9.1); ROC curves were produced and compared using customized STATA modules. (Pepe 2003).

Statistical Analysis of ELISA Data: Candidate protein biomarker concentrations in cord blood measured by ELISA experiments in control subjects without sepsis (n=77), and subjects with confirmed sepsis (n=5) were log transformed before subjecting them to statistical analysis. Independent pair-wise comparisons of log-transformed protein concentrations between control vs. sepsis were performed using one-way analysis of variance (ANOVA) test. For presentation, we transformed the average log value back to original units (harmonic mean), and applied the Bonferroni correction to account for multiple comparisons. Based on results from individual protein comparisons, we evaluated the classification performance of several different combinations of 2, 3 or 4 proteins using logistic regression models. Receiver operating characteristic (ROC) curves were computed based on the risk scores from each of the multi-protein models.

Descriptive and inferential statistics were computed using SAS software (v9.1); ROC curves were produced and compared using customized STATA modules. (Pepe 2003).

### Results

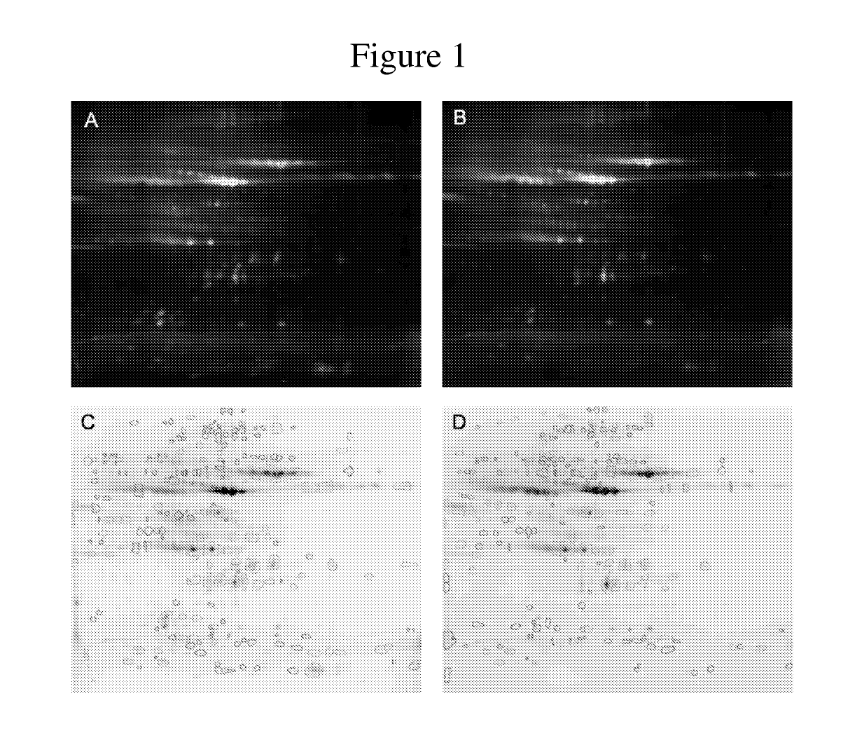

Proteomic changes in cord blood proteome in neonatal sepsis: 2-dimensional gel electrophoresis analysis: Cord blood (CB) from control, suspected sepsis (SS), and confirmed sepsis (CS) subjects was subjected to affinity purification to remove high abundance serum proteins. Depleted CBS from control, SS, and CS subjects were labeled with Cy5, Cy3, and Cy2 dyes, respectively. Labeled samples were resolved on a 2D gel. FIGS. 1A and 1B show DIGE gel images of CBS from control (red) vs. SS (green) and control (red) vs. CS (green), respectively. Spots that are differentially expressed between SS vs. control (FIG. 1C) and CS vs. control (FIG. 1D) were determined using Phoretix 2D evolution software. Spot intensities in difference maps (FIGS. 1C and 1D) were normalized based on total spot volume. Spots in difference maps that are ≧2 fold down regulated were highlighted in red and ≧2 fold up regulated were highlighted in green.

Conclusion: 2-D gel analysis identified differential expression of multiple proteins in the cord blood of neonatal sepsis subjects.

Cord Blood Proteome: A total of 670 proteins with at least two unique peptide (p≧0.8) matches were identified from all 2-DLC mass spectrometry experiments. Cord blood proteins are ranked according to the decreasing order of spectral counts and shown in Supplemental Table 1 (column No. 5). Functional annotation of cord blood proteome was performed using Gene Ontology (GO) annotations from DAVID bioinformatics resource (Dennis 2003). Proteins with metabolic (21%), immune response (10%), transport (10%), and developmental (7%) functions constituted a majority of the cord blood proteome.

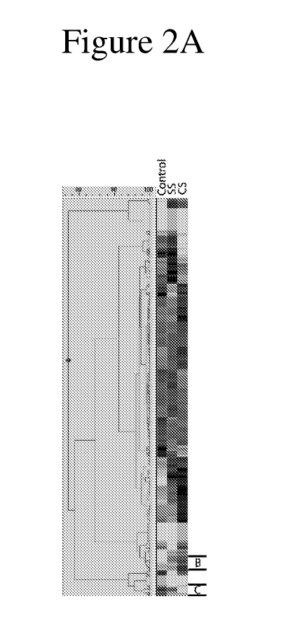

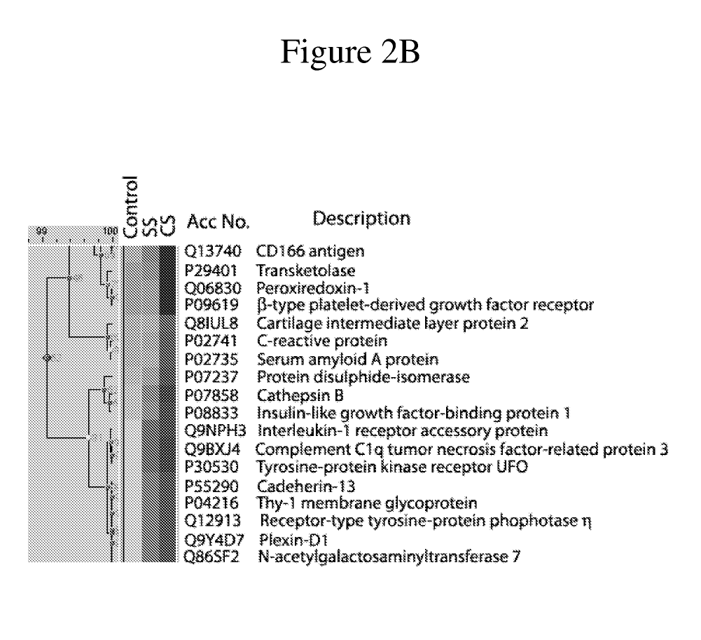

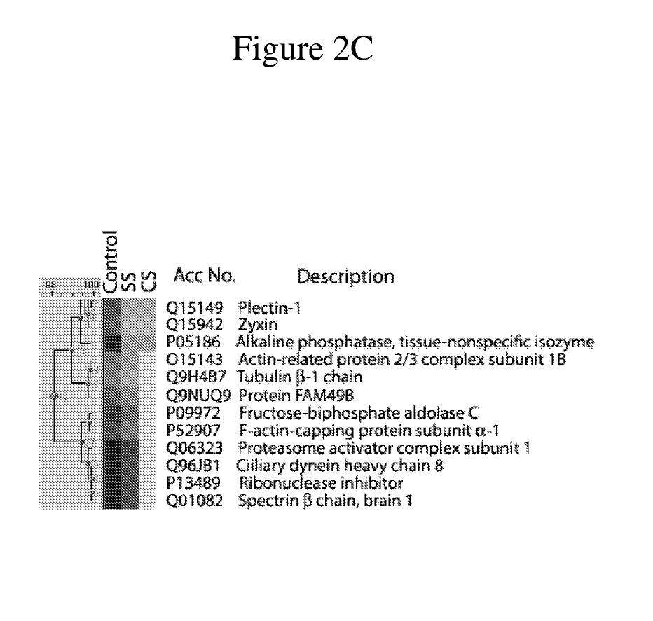

Clustering of differentially expressed proteins in cord blood proteome in neonatal sepsis: Total number of MS/MS spectra matched to a protein is directly related to its abundance in complex mixtures. (Liu 2004). Global protein expression changes in CB between control, SS, and CS subjects are visualized using GeneMaths software (version 1.5, Applied Maths, Austin, Tex.). Spectral counts of proteins with at least two peptide identifications (p≧0.8) in one of the samples were individually mean normalized and analyzed by GeneMaths software. Proteins with similar expression changes between samples were hierarchically clustered using Euclidean distance learning method with 200 simulations (FIG. 2A). Representative protein clusters with proteins that are up regulated in CS and control samples are shown in FIG. 2B and FIG. 2C, respectively.

Conclusion: Visualization of cord blood proteome using hierarchical clustering demonstrated specific clusters of proteins over expressed in neonatal sepsis subjects.

Cord blood biomarkers for neonatal sepsis identified by 2-dimensional liquid chromatography and tandem mass spectrometry (2D LC-MS-MS): CB samples from control and confirmed sepsis (CS) samples were subjected to 2-DLC based tandem mass spectrometry followed by label-free quantification. CB proteins that passed label-free quantification with a p value of ≦0.05 and a fold change of ≧+2.0 were considered as significantly differentially expressed between control and neonatal sepsis subjects (Table 1). Biological function annotation for differentially expressed proteins in Table 1 was performed using Bioinformatics Harvester. Table 1 below lists differentially expressed cord blood proteins between control and neonatal sepsis samples with their Swiss-Prot accession number, description, fold change, and p-value. Proteins were grouped according to their biological function.

TABLE 1
Cord Blood Biomarkers of Neonatal Sepsis

| | Swiss- | | CS vs. Control | |
|---|---|---|---|---|
| Biological | Prot Acc. | | Fold | |
| Function | No | Description | Change | P Value |
| | P02741 | C-reactive protein precursor (SEQ ID NO: 1) | 6.6 | <0.0001 |
| | Q9NPH3 | Interleukin-1 receptor accessory protein precursor | 6.4 | 0.0117 |
| | | (SEQ ID NO: 2) | | |
| | P05231 | Interleukin-6 precursor (SEQ ID NO: 3) | 5.5 | 0.0246 |
| | Q01638 | Interleukin-1 receptor-like 1 precursor (SEQ ID NO: 4) | 5.5 | 0.0246 |
| | P02735 | Serum amyloid A protein precursor (SEQ ID NO: 5) | 3.8 | 0.0179 |
| | O43866 | CD5 antigen-like precursor (SEQ ID NO: 6) | 3.4 | 0.0095 |
| Inflammation | P61769 | Beta-2-microglobulin precursor (SEQ ID NO: 7) | 2.5 | 0.0001 |
| and Immune | P13727 | Bone-marrow proteoglycan precursor (SEQ ID NO: 8) | 2.5 | 0.0039 |
| response | Q13228 | Selenium-binding protein 1 (SEQ ID NO: 9) | 2.4 | 0.0231 |
| modulators | P18428 | Lipopolysaccharide-binding protein precursor (SEQ ID | 2.4 | <0.0001 |
| | | NO: 10) | | |
| | Q6UVK1 | Chondroitin sulfate proteoglycan 4 precursor (SEQ ID | 2.3 | 0.0104 |
| | | NO: 11) | | |
| | P10451 | Osteopontin precursor (SEQ ID NO: 12) | 2.2 | 0.0022 |
| | P52566 | Rho GDP-dissociation inhibitor 2 (SEQ ID NO: 13) | −2.2 | 0.0189 |
| | P00918 | Carbonic anhydrase 2 (SEQ ID NO: 14) | −3.1 | 0.0234 |
| | P80188 | Neutrophil gelatinase-associated lipocalin precursor | −3.5 | 0.0087 |
| | | (SEQ ID NO: 15) | | |
| | P29400 | Collagen alpha-5(IV) chain precursor (SEQ ID NO: 16) | 7.2 | 0.0056 |
| | P29279 | Connective tissue growth factor precursor (SEQ ID | 7.2 | 0.0056 |
| | | NO: 17) | | |
| Extracellular | P09603 | Macrophage colony-stimulating factor 1 precursor | 5.5 | 0.0246 |
| Matrix, | | (SEQ ID NO: 18) | | |
| Matricellular, | Q99435 | Protein kinase C-binding protein NELL2 precursor | 4.5 | 0.0019 |
| and | | (SEQ ID NO: 19) | | |
| Cytoskeletal | Q9UMX5 | Neudesin precursor (SEQ ID NO: 20) | 4.5 | 0.0168 |
| | P07237 | Protein disulfide-isomerase precursor (SEQ ID NO: 21) | 4 | 0.0317 |
| | P07998 | Ribonuclease pancreatic precursor (SEQ ID NO: 22) | 3.9 | 0.0007 |
| | P80370 | Delta-like protein precursor (SEQ ID NO: 23) | 3.8 | 0.0034 |
| | P10645 | Chromogranin-A precursor (SEQ ID NO: 24) | 3.6 | 0.0002 |
| | Q99983 | Osteomodulin precursor (SEQ ID NO: 25) | 3.5 | 0.0318 |
| | P08123 | Collagen alpha-2(I) chain precursor (SEQ ID NO: 26) | 3.2 | 0.0004 |
| | Q07954 | Prolow-density lipoprotein receptor-related protein 1 | 3.1 | 0.0005 |
| | | precursor (SEQ ID NO: 27) | | |
| | P11047 | Laminin subunit gamma-1 precursor (SEQ ID NO: 28) | 2.8 | 0.0422 |
| | P07942 | Laminin subunit beta-1 precursor (SEQ ID NO: 29) | 2.4 | 0.001 |
| | P02458 | Collagen alpha-1(II) chain precursor (SEQ ID NO: 30) | 2.4 | 0.0231 |
| | P01033 | Metalloproteinase inhibitor 1 precursor (SEQ ID NO: 31) | 2.3 | 0.0169 |
| | Q92520 | Protein FAM3C precursor (SEQ ID NO: 32) | 2.2 | 0.0418 |
| | P12814 | Alpha-actinin-1 (SEQ ID NO: 33) | −3.3 | 0.0143 |
| | P52907 | F-actin-capping protein subunit alpha-1 (SEQ ID | −6.7 | 0.0083 |
| | | NO: 34) | | |
| | P15144 | Aminopeptidase N (SEQ ID NO: 35) | 13.4 | <0.0001 |
| | P08833 | Insulin-like growth factor-binding protein 1 precursor | 10.1 | <0.0001 |
| | | (SEQ ID NO: 36) | | |
| | Q9BY67 | Cell adhesion molecule 1 precursor (SEQ ID NO: 37) | 8.1 | 0.0027 |
| | P07858 | Cathepsin B precursor (SEQ ID NO: 38) | 5.5 | 0.0046 |
| | Q93063 | Exostosin-2 (SEQ ID NO: 39) | 5.5 | 0.0246 |
| | P07339 | Cathepsin D precursor (SEQ ID NO: 40) | 3.8 | <0.0001 |
| Development | Q9UM47 | Neurogenic locus notch homolog protein 3 precursor | 3.4 | 0.0095 |
| and Apoptosis | | (SEQ ID NO: 41) | | |
| | Q15828 | Cystatin-M precursor (SEQ ID NO: 42) | 2.9 | 0.0031 |
| | Q99784 | Noelin precursor (SEQ ID NO: 43) | 2.9 | 0.0491 |
| | P18065 | Insulin-like growth factor-binding protein 2 precursor | 2.6 | <0.0001 |
| | | (SEQ ID NO: 44) | | |
| | P14625 | Endoplasmin precursor (SEQ ID NO: 45) | 2.3 | 0.0169 |
| | Q8NBP7 | Proprotein convertase subtilisin/kexin type 9 precursor | 2.2 | 0.0046 |
| | | (SEQ ID NO: 46) | | |
| | P35858 | Insulin-like growth factor-binding protein complex acid | −2.3 | 0.0016 |
| | | labile chain precursor (SEQ ID NO: 47) | | |
| ERM | P15311 | Ezrin (SEQ ID NO: 48) | 5.5 | 0.0046 |
| | P07148 | Fatty acid-binding protein, liver (SEQ ID NO: 49) | 8.1 | 0.0027 |
| | Q8IZF2 | Probable G-protein coupled receptor 116 precursor | 6.5 | 0.0012 |
| | | (SEQ ID NO: 50) | | |
| | Q12884 | Seprase (SEQ ID NO: 51) | 6.4 | 0.0117 |
| | Q8WWZ8 | Oncoprotein-induced transcript 3 protein precursor | 4.5 | 0.0168 |
| | | (SEQ ID NO: 52) | | |
| | Q9Y4L1 | Hypoxia up-regulated protein 1 precursor (SEQ ID | 3.5 | 0.0318 |
| | | NO: 53) | | |
| | O43493 | Trans-Golgi network integral membrane protein 2 | 3.5 | 0.0318 |
| | | precursor (SEQ ID NO: 54) | | |
| Proteins of | P29401 | Transketolase (SEQ ID NO: 55) | 3.4 | 0.0173 |
| miscellaneous | P10586 | Receptor-type tyrosine-protein phosphatase F | 2.9 | 0.0491 |
| class or | | precursor (SEQ ID NO: 56) | | |
| unknown | P05362 | Intercellular adhesion molecule 1 precursor (SEQ ID | 2.8 | 0.006 |
| | | NO: 57) | | |
| | P01130 | Low-density lipoprotein receptor precursor (SEQ ID | 2.8 | 0.006 |
| | | NO: 58) | | |
| | P11021 | 78 kDa glucose-regulated protein precursor (SEQ ID | 2.6 | 0.0049 |
| | | NO: 59) | | |
| | Q8TDY8 | Neighbor of punc e11 precursor (SEQ ID NO: 60) | 2.3 | 0.0455 |
| | P33908 | Mannosyl-oligosaccharide 1,2-alpha-mannosidase IA | 2.2 | 0.0418 |
| | | (SEQ ID NO: 61) | | |
| | P14618 | Pyruvate kinase isozymes M1/M2 (SEQ ID NO: 62) | −5 | 0.007 |
| | P31948 | Stress-induced-phosphoprotein 1 (SEQ ID NO: 63) | −5.3 | 0.0323 |

Conclusion: 2D-LC MS-MS analysis identified differential abundance of 60 potential biomarkers of neonatal sepsis in cord blood that are statistically significant.

Validation of potential neonatal sepsis biomarkers using enzyme linked immunosorbent assays: A total of 10 significantly differentially expressed proteins from the 2-DLC study were cross validated on a cohort of 77 control and 5 neonatal sepsis subjects, using ELISA. Measured protein concentrations were log-transformed and compared in a pair-wise between control and sepsis groups, using an ANOVA test. Proteins that passed the comparison with a p-value ≦0.05 are shown in Table 2 below. The mean concentration of each protein in respective sample groups was determined by computing the harmonic mean of protein concentrations (ng/ml) measured by ELISA (shown in Table 2).

TABLE 2
Validation of potential neonatal sepsis biomarkers with ELISA

| | | | Control, | | Confirmed | |
|---|---|---|---|---|---|---|
| | | | No Sepsis | Confirmed | Sepsis vs. | |
| Accession | | | (n = 77) | Sepsis (n = 5) | Control, No | |
| (SEQ ID | | | Geometric | Geometric | Sepsis | |
| NO) | ID | Protein | Mean ng/ml | Mean ng/ml | p value | AUROC |
| P08833 | IBP1 | Insulin-like growth | 74.3 | 1671.2 | 0.0061 | 0.918 |
| (SEQ ID | | factor-binding protein 1 | | | | |
| NO: 36) | | | | | | |
| P05231 | IL6 | Interleukin-6 | 0.7 | 401.1 | 0.0009 | 0.790 |
| (SEQ ID | | | | | | |
| NO: 3) | | | | | | |
| P02741 | CRP | C-reactive protein | 248.2 | 4910.4 | 0.0030 | 0.862 |
| (SEQ ID | | | | | | |
| NO: 1) | | | | | | |
| P61769 | B2MG | Beta-2-microglobulin | 2434.2 | 4410.2 | 0.0082 | 0.835 |
| (SEQ ID | | | | | | |
| NO: 7) | | | | | | |
| P07858 | CATB | Cathepsin B | 162.2 | 487.8 | 0.0012 | 0.805 |
| (SEQ ID | | | | | | |
| NO: 38) | | | | | | |
| Q15828 | CYTM | Cystatin-M | 154.5 | 211.3 | 0.2295 | 0.600 |
| (SEQ ID | | | | | | |
| NO: 42) | | | | | | |
| P18065 | IBP2 | Insulin-like growth | 142.5 | 212.7 | 0.0910 | 0.719 |
| (SEQ ID | | factor-binding protein 2 | | | | |
| NO: 44) | | | | | | |
| P14780 | MMP9 | Matrix | 178.3 | 54.7 | 0.0052 | 0.881 |
| (SEQ ID | | metalloproteinase-9 | | | | |
| NO: 64) | | | | | | |
| P01033 | TIMP1 | Metalloproteinase | 278.0 | 473.0 | 0.0131 | 0.761 |
| (SEQ ID | | inhibitor 1 | | | | |
| NO: 31) | | | | | | |
| P02763 | A1AG1 | Alpha-1-acid | 32225.8 | 285987.5 | 0.0291 | 0.783 |
| (SEQ ID | | glycoprotein 1 | | | | |
| NO: 65) | | | | | | |

Conclusion: ELISA analysis of potential biomarkers on individual samples confirmed the differential expression of candidate proteins observed by 2D-LC-MS-MS analysis.

### REFERENCES

Clark R, Powers R, White R, Bloom B, Sanchez P, Benjamin D K, Jr. Nosocomial infection in the NICU: a medical complication or unavoidable problem? J Perinatol 2004; 24(6):382-8.
Lopez Sastre J B, Coto Cotallo D, Fernandez Colomer B. Neonatal sepsis of nosocomial origin: an epidemiological study from the “Grupo de Hospitales Castrillo”. J Perinat Med 2002; 30(2):149-57.
Craft A, Finer N. Nosocomial coagulase negative staphylococcal (CoNS) catheter-related sepsis in preterm infants: definition, diagnosis, prophylaxis, and prevention. J Perinatol 2001; 21(3):186-92.
Zafar N, Wallace C M, Kieffer P, Schroeder P, Schootman M, Hamvas A. Improving survival of vulnerable infants increases neonatal intensive care unit nosocomial infection rate. Arch Pediatr Adolesc Med 2001; 155(10):1098-104.
Ferguson J K, Gill A. Risk-stratified nosocomial infection surveillance in a neonatal intensive care unit: report on 24 months of surveillance. J Paediatr Child Health 1996; 32(6):525-31.
Hentschel J, de Veer I, Gastmeier P, Ruden H, Obladen M. Neonatal nosocomial infection surveillance: incidences by site and a cluster of necrotizing enterocolitis. Infection 1999; 27(4-5):234-8.
Drews M B, Ludwig A C, Leititis J U, Daschner F D. Low birth weight and nosocomial infection of neonates in a neonatal intensive care unit. J Hosp Infect 1995; 30(1):65-72.
Berger A, Salzer H R, Weninger M, Sageder B, Aspock C. Septicaemia in an Austrian neonatal intensive care unit: a 7-year analysis. Acta Paediatr 1998; 87(10):1066-9.
Horbar J D, Rogowski J, Plsek P E, et al. Collaborative quality improvement for neonatal intensive care. NIC/Q Project Investigators of the Vermont Oxford Network. Pediatrics 2001; 107(1):14-22.
Nagata E, Brito A S, Matsuo T. Nosocomial infections in a neonatal intensive care unit: incidence and risk factors. Am J Infect Control 2002; 30(1):26-31.
Gaynes R P, Edwards J R, Jarvis W R, Culver D H, Tolson J S, Martone W J. Nosocomial infections among neonates in high-risk nurseries in the United States. National Nosocomial Infections Surveillance System. Pediatrics 1996; 98(3 Pt 1):357-61.
Fenyo D, Beavis R C. A method for assessing the statistical significance of mass spectrometry-based protein identifications using general scoring schemes. Anal Chem 2003; 75(4):768-74.
Nesvizhskii A I, Keller A, Kolker E, Aebersold R. A statistical model for identifying proteins by tandem mass spectrometry. Anal Chem 2003; 75(17):4646-58.
Liu H, Sadygov R G, Yates J R, 3rd. A model for random sampling and estimation of relative protein abundance in shotgun proteomics. Anal Chem 2004; 76(14):4193-201.
Old W M, Meyer-Arendt K, Aveline-Wolf L, et al. Comparison of label-free methods for quantifying human proteins by shotgun proteomics. Mol Cell Proteomics 2005; 4(10):1487-502. Epub 2005 Jun. 23.
Gravett M G, Thomas A, Schneider K A, et al. Proteomic analysis of cervical-vaginal fluid: identification of novel biomarkers for detection of intra-amniotic infection. J Proteome Res 2007; 6(1):89-96.
Nagalla S R, Canick J A, Jacob T, et al. Proteomic analysis of maternal serum in down syndrome: identification of novel protein biomarkers. J Proteome Res 2007; 6(4):1245-57.
Pereira L, Reddy A P, Jacob T, et al. Identification of novel protein biomarkers of preterm birth in human cervical-vaginal fluid. J Proteome Res 2007; 6(4):1269-76.
Zybailov B, Mosley A L, Sardiu M E, Coleman M K, Florens L, Washburn M P. Statistical analysis of membrane proteome expression changes in Saccharomyces cerevisiae. J Proteome Res 2006; 5(9):2339-47.
Pepe M S. The statistical evaluation of medical tests for classification and prediction. Oxford: Oxford University Press; 2003.
Dennis G, Jr., Sherman B T, Hosack D A, et al. DAVID: Database for Annotation, Visualization, and Integrated Discovery. Genome Biol 2003; 4(5):P3.
Stoll B J, Hansen N, Fanaroff A A, et al. Late onset sepsis in very low birth weight neonates; the experience of National Institute of Child Health and Human Development Neonatal Research Network. J Pediatr 1996; 110:285-91.

1. A method for diagnosis of neonatal sepsis in a mammalian subject comprising:
(a) testing in a sample of biological fluid obtained from said subject the level of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), relative to the level in normal biological fluid or biological fluid known to be indicative of neonatal sepsis; and
(b) diagnosing said subject with neonatal sepsis if said level shows a statistically significant difference relative to the level in said normal biological fluid, or does not show a statistically significant difference relative to the level in said biological fluid known to be indicative of neonatal sepsis.

2. The method of claim 1, wherein the subject is a human patient.

3. The method of claim 2, wherein said testing is implemented using an apparatus adapted to determine the level of said proteins.

4. The method of claim 2, wherein said testing is performed by using a software program executed by a suitable processor.

5. The method of claim 4, wherein the program is embodied in software stored on a tangible medium.

6. The method of claim 5, wherein the tangible medium is selected from the group consisting of a CD-ROM, a floppy disk, a hard drive, a DVD, and a memory associated with the processor.

7. The method of any one of claims 2 to 6, further comprising the step of preparing a report recording the results of said testing or the diagnosis.

8. The method of claim 7, wherein said report is recorded or stored on a tangible medium.

9. The method of claim 8, wherein the tangible medium is paper.

10. The method of claim 8, wherein the tangible medium is selected from the group consisting of a CD-ROM, a floppy disk, a hard drive, a DVD, and a memory associated with the processor.

11. The method of any one of claims 2 to 6, further comprising the step of communicating the results of said diagnosis to an interested party.

12. The method of claim 11, wherein the interested party is the patient or the attending physician.

13. The method of claim 11, wherein the communication is in writing, by email, or by telephone.

14. The method of claim 1, wherein said biological fluid is selected from the group consisting of cord blood, cerebrospinal fluid, and neonatal serum.

15. The method of claim 14, wherein said biological fluid is cord blood.

16. The method of claim 2, wherein said diagnosis is determined within 24 hours of birth.

17. The method of claim 2 comprising testing the level of at least two of said proteins.

18. The method of claim 2 comprising testing the level of at least three of said proteins.

19. The method of claim 2 comprising testing the level of at least four of said proteins.

20. The method of claim 2 comprising testing the level of proteins Insulin-like growth factor-binding protein 1 precursor (SEQ ID NO:36), Interleukin-6 precursor (SEQ ID NO:3), C-reactive protein precursor (SEQ ID NO:1), Beta-2-microglobulin precursor (SEQ ID NO:7), Cathepsin B precursor (SEQ ID NO:38), Cystatin-M precursor (SEQ ID NO:42), Insulin-like growth factor-binding protein 2 precursor (SEQ ID NO:44), Matrix metalloproteinase-9 (SEQ ID NO:64), Metalloproteinase inhibitor 1 precursor (SEQ ID NO:31), and Alpha-1-acid glycoprotein 1 (SEQ ID NO:65), and diagnosing said subject with neonatal sepsis, if one or more of said tested proteins shows a significant difference in the cord blood sample relative to normal cord blood.

21. The method of claim 20 comprising diagnosing said subject with neonatal sepsis, if all of said tested proteins show a significant difference in the cord blood sample relative to normal cord blood.

22. The method of claim 2 wherein said level is determined by an immunoassay.

23. The method of claim 2 wherein level is determined by mass spectrometry.

24. The method of claim 2 wherein level is determined using a protein array.

25. Use of any one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), in the manufacture of a proteomic profile of a biological fluid for the early diagnosis of neonatal sepsis in a subject.

26. The use of claim 25 wherein the subject is a human patient.

27. The use of claim 25 wherein the biological fluid is selected from the group consisting of cord blood, neonatal serum and cerebrospinal fluid.

28. The use of claim 26 wherein the proteomic profile comprises information of the level of said proteins and wherein the diagnosis of said subject with neonatal sepsis is made if one or more of said tested proteins shows a significant difference in the biological fluid sample relative to normal biological fluid.

29. The use of claim 26, wherein said diagnosis is determined within 24 hours of birth.

30. The use of claim 26 wherein the proteomic profile comprises information of the level of at least two of said proteins.

31. The use of claim 26 wherein the proteomic profile comprises information of the level of at least three of said proteins.

32. The use of claim 26 wherein the proteomic profile comprises information of the level of at least four of said proteins.

33. The use of claim 2 wherein the proteomic profile comprises information of the level of proteins Insulin-like growth factor-binding protein 1 precursor (SEQ ID NO:36), Interleukin-6 precursor (SEQ ID NO:3), C-reactive protein precursor (SEQ ID NO:1), Beta-2-microglobulin precursor (SEQ ID NO:7), Cathepsin B precursor (SEQ ID NO:38), Cystatin-M precursor (SEQ ID NO:42), Insulin-like growth factor-binding protein 2 precursor (SEQ ID NO:44), Matrix metalloproteinase-9 (SEQ ID NO:64), Metalloproteinase inhibitor 1 precursor (SEQ ID NO:31), and Alpha-1-acid glycoprotein 1 (SEQ ID NO:65), and wherein the diagnosis of said subject with neonatal sepsis is made if one or more of said tested proteins shows a significant difference in the biological fluid sample relative to normal biological fluid.

34. The use of claim 33 wherein the diagnosis of said subject with neonatal sepsis is made if all of said tested proteins show a significant difference in the biological fluid sample relative to normal biological fluid.

35. The use of claim 26 wherein said level is determined by an immunoassay.

36. The use of claim 26 wherein said level is determined by mass spectrometry.

37. The use of claim 26 wherein said level is determined using a protein array.

38. An immunoassay kit comprising antibodies and reagents for the detection of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63).

39. An immunoassay kit comprising antibodies and reagents for the detection of one or more proteins selected from the group consisting of Insulin-like growth factor-binding protein 1 precursor (SEQ ID NO:36), Interleukin-6 precursor (SEQ ID NO:3), C-reactive protein precursor (SEQ ID NO:1), Beta-2-microglobulin precursor (SEQ ID NO:7), Cathepsin B precursor (SEQ ID NO:38), Cystatin-M precursor (SEQ ID NO:42), Insulin-like growth factor-binding protein 2 precursor (SEQ ID NO:44), Matrix metalloproteinase-9 (SEQ ID NO:64), Metalloproteinase inhibitor 1 precursor (SEQ ID NO:31), and Alpha-1-acid glycoprotein 1 (SEQ ID NO:65).

40. The immunoassay kit of claim 38 comprising antibodies and reagents for the detection of all of said proteins.

41. A report comprising the results of and/or diagnosis based on a test comprising
(a) testing in a sample of biological fluid obtained from said subject the level of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), relative to the level in normal biological fluid or biological fluid known to be indicative of neonatal sepsis; and
(b) diagnosing said subject with neonatal sepsis if said level shows a statistically significant difference relative to the level in said normal biological fluid, or does not show a statistically significant difference relative to the level in said biological fluid known to be indicative of neonatal sepsis.

42. A tangible medium storing the results of and/or diagnosis based on a test comprising
(a) testing in a sample of biological fluid obtained from said subject the level of one or more proteins selected from the group consisting of C-reactive protein precursor (SEQ ID NO:1), Interleukin-1 receptor accessory protein precursor (SEQ ID NO:2), Interleukin-6 precursor (SEQ ID NO:3), Interleukin-1 receptor-like 1 precursor (SEQ ID NO:4), Serum amyloid A protein precursor (SEQ ID NO:5), CD5 antigen-like precursor (SEQ ID NO:6), Beta-2-microglobulin precursor (SEQ ID NO:7), Bone-marrow proteoglycan precursor (SEQ ID NO:8), Selenium-binding protein 1 (SEQ ID NO:9), Lipopolysaccharide-binding protein precursor (SEQ ID NO:10), Chondroitin sulfate proteoglycan 4 precursor (SEQ ID NO:11), Osteopontin precursor (SEQ ID NO:12), Rho GDP-dissociation inhibitor 2 (SEQ ID NO:13), Carbonic anhydrase 2 (SEQ ID NO:14), Neutrophil gelatinase-associated lipocalin precursor (SEQ ID NO:15), Collagen alpha-5(IV) chain precursor (SEQ ID NO:16), Connective tissue growth factor precursor (SEQ ID NO:17), Macrophage colony-stimulating factor 1 precursor (SEQ ID NO:18), Protein kinase C-binding protein NELL2 precursor (SEQ ID NO:19), Neudesin precursor (SEQ ID NO:20), Protein disulfide-isomerase precursor (SEQ ID NO:21), Ribonuclease pancreatic precursor (SEQ ID NO:22), Delta-like protein precursor (SEQ ID NO:23), Chromogranin-A precursor (SEQ ID NO:24), Osteomodulin precursor (SEQ ID NO:25), Collagen alpha-2(I) chain precursor (SEQ ID NO:26), Prolow-density lipoprotein receptor-related protein 1 precursor (SEQ ID NO:27), Laminin subunit gamma-1 precursor (SEQ ID NO:28), Laminin subunit beta-1 precursor (SEQ ID NO:29), Collagen alpha-1(II) chain precursor (SEQ ID NO:30), Metalloproteinase inhibitor 1 precursor (SEQ ID NO:31), Protein FAM3C precursor (SEQ ID NO:32), Alpha-actinin-1 (SEQ ID NO:33), F-actin-capping protein subunit alpha-1 (SEQ ID NO:34), Aminopeptidase N (SEQ ID NO:35), Insulin-like growth factor-binding protein 1 precursor (SEQ ID NO:36), Cell adhesion molecule 1 precursor (SEQ ID NO:37), Cathepsin B precursor (SEQ ID NO:38), Exostosin-2 (SEQ ID NO:39), Cathepsin D precursor (SEQ ID NO:40), Neurogenic locus notch homolog protein 3 precursor (SEQ ID NO:41), Cystatin-M precursor (SEQ ID NO:42), Noelin precursor (SEQ ID NO:43), Insulin-like growth factor-binding protein 2 precursor (SEQ ID NO:44), Endoplasmin precursor (SEQ ID NO:45), Proprotein convertase subtilisin/kexin type 9 precursor (SEQ ID NO:46), Insulin-like growth factor-binding protein complex acid labile chain precursor (SEQ ID NO:47), Ezrin (SEQ ID NO:48), Fatty acid-binding protein, liver (SEQ ID NO:49), Probable G-protein coupled receptor 116 precursor (SEQ ID NO:50), Seprase (SEQ ID NO:51), Oncoprotein-induced transcript 3 protein precursor (SEQ ID NO:52), Hypoxia up-regulated protein 1 precursor (SEQ ID NO:53), Trans-Golgi network integral membrane protein 2 precursor (SEQ ID NO:54), Transketolase (SEQ ID NO:55), Receptor-type tyrosine-protein phosphatase F precursor (SEQ ID NO:56), Intercellular adhesion molecule 1 precursor (SEQ ID NO:57), Low-density lipoprotein receptor precursor (SEQ ID NO:58), 78 kDa glucose-regulated protein precursor (SEQ ID NO:59), Neighbor of punc e11 precursor (SEQ ID NO:60), Mannosyl-oligosaccharide 1,2-alpha-mannosidase IA (SEQ ID NO:61), Pyruvate kinase isozymes M1/M2 (SEQ ID NO:62), Matrix metalloproteinase-9 (SEQ ID NO:64), Alpha-1-acid glycoprotein 1 (SEQ ID NO:65), and Stress-induced-phosphoprotein 1 (SEQ ID NO:63), relative to the level in normal biological fluid or biological fluid known to be indicative of neonatal sepsis; and
(b) diagnosing said subject with neonatal sepsis if said level shows a statistically significant difference relative to the level in said normal biological fluid, or does not show a statistically significant difference relative to the level in said biological fluid known to be indicative of neonatal sepsis.
